SEQUENCE LISTING (70 sequences)

SEQ ID NO: 1 - protein, 35 aa, Mus musculus
MLPAVKTVEAEEEYAEDCPELVPIETKNQEEENLD

SEQ ID NO: 2 - protein, 37 aa, Homo sapiens
MLPAVGSVDEEEDPAEEDCPELVPIETTQSEEEEKSG

SEQ ID NO: 3 - protein, 19 aa, Danio rerio
MEDEDECPELVPIKEKPSG

SEQ ID NO: 4 - protein, 17 aa, Mus musculus
AEEEYAEDCPELVPIET

SEQ ID NO: 5 - protein, 13 aa, Homo sapiens
EDEDECPELVPIK

SEQ ID NO: 6 - protein, 14 aa, Danio rerio
PAEEDCPELVPIRT

SEQ ID NO: 7 - protein, 395 aa, Homo sapiens
MLPAVGSVDEEEDPAEEDCPELVPIETTQSEEEEKSGLGAKIPVTIITGYLGAGKTTLLNYILTEQHSKRVAVILNESGEGSALEKSLAVSQGGELYEEWLELRNGCLCCSVKDNGLRAIENLMQKKGKFDDILLETTGLADPGAVTSMFWVDAELGSDIYLDGIITIVDSKYGLKHLTEEKPDGLINEATRQVALADIILINKTDLVPEEDVKKLRTTIRSINGLGQILETQRSRVDLSNVLDLHAFDSLSGISLQKKLQHVPGTQPHLDQSIVTITFEVPGNAKEEHLNMFIQNLLWEKNVRNKDNHCMEVIRLKGLVSIKDKSQQVIVQGVHELYDLEETPVSWKDDTERTNRLVLIGRNLDKDILKQLFIATVTETEKQWTTHFKEDQVCT

SEQ ID NO: 8 - protein, 395 aa, Homo sapiens
MLPAVGSVDEEEDPAEEDCPELVPIETTQSEEEEKSGLGAKIPVTIITGYLGAGKTTLLNYILTEQHSKRVAVILNESGEGSALEKSLAVSQGGELYEEWLELRNGCLCCSVKDNGLRAIENLMQKKGKFDDILLETTGLADPGAVASMFWVDAELGSDIYLDGIITIVDSKYGLKHLTEEKPDGLINEATRQVALADIILINKTDLVPEEDVKKLRTTIRSINGLGQILETQRSRVDLSNVLDLHAFDSLSGISLQKKLQHVPGTQPHLDQSIVTITFEVPGNAKEEHLNMFIQNLLWEKNVRNKDNHCMEVIRLKGLVSIKDKSQQVIVQGVHELYDLEETPVSWKDDTERTNRLVLIGRNLDKDILKQLFIATVTETEKQWTTHFKEDQVCT

SEQ ID NO: 9 - protein, 395 aa, Homo sapiens
MLPAVGSVDEEEDPAEEDCPELVPIETTQSEEEEKSGLGAKIPVTIITGYLGAGKTTLLNYILTEQHSKRVAVILNESGEGSALEKSLAVSQGGELYEEWLELRNGCLCCSVKDNGLRAIENLMQKKGKFDDILLETTGLADPGAVASMFWVDAELGSDIYLDGIITIVDSKYGLKHLTEEKPDGLINEATRQVALADIILELRNGCLCCSVKDNGLRAIENLMQKKGKFDDILLETTGLADPGAVASMFWVDAELGSDIYLDGIITIVDSKYGLKHLTEEKPDGLINEATRQVALADIIKNVRNKDNHCMEVIRLKGLVSIKDKSQQVIVQGVHELCDLEETPVSWKDDTERTNRLVLIGRNLDKDILKQLFIATVTETEKQWTTHFKEDQVCT

SEQ ID NO: 10 - protein, 399 aa, Homo sapiens
MLPAVGSADEEEDPAEEDCPELVPIETTQSEEEEKSGLGAKIPVTIITGYLGAGKTTLLNYILTEQHSKRVAVILNEFGEGSALEKSLAVSQGGELYEEWLELRNGCLCCSVZNGCKDSGLRAIENLMQKKGKFDYILLETTGLADPGAVASMFWVDAELGSDIYLDGIITIVDSKYGLKHLTEEKPDGLINEATRQVALADAILINKTDLVPEEDVKKLRTTIRSINGLGQILETQRSRVDLSNVLDLHAFDSLSGISLQKKLQHVPGTQPHLDQSIVTITFEVPGNAKEEHLNMFIQNLLWEKNVRNKDNHCMEVIRLKGLVSIKDKSQQVIVQGVHELYDLEETPVSWKDDTERTNRLVLLGRNLDKDILKQLFIATVTETEKQWTTRFQEDQVCT

SEQ ID NO: 11 - protein, 395 aa, Homo sapiens
MLPAVGSADEEEDPAEEDCPELVPMETTQSEEEEKSGLGAKIPVTIITGYLGAGKTTLLNYILTEQHSKRVAVILNEFGEGSALEKSLAVSQGGELYEEWLELRNGCLCCSVKDNGLRAIENLMQKKGKFDYILLETTGLADPGAVASMFWVDAELGSDIYLDGIITIVDSKYGLKHLAEEKPDGLINEATRQVALADAILINKTDLVPEEDVKKLRATIRSINGLGQILETQRSRVDLSNVLDLHAFDSLSGISLQKKLQHVPGTQPHLDQSIVTITFEVPGNAKEEHLNMFIQNLLWEKNVRNKDNHCMEVIRLKGLVSIKDKSQQVIVQGVHELYDLEETPVSWKDDTERTNRLVLLGRNLDKDILKQLFIATVTETEKQWTTHFKEDQVCT

SEQ ID NO: 12 - protein, 366 aa, synthetic construct
  REGION 1..366: note = Consensus sequence
MLPAVCSVDEEEDPAEEDCPELVPIETTQSEEEEKSGLGAKPVTTCYLGAGKTTLLNYLTEQHSKRVAVLNESGEGSALEKSLAVSQCCELYEEWLELRNGCLCCSVKDNGLRAENLMQKKGKFDDLLETTGLADPGAVASMFWVDAELGSDYLDGTVDSKYGLKHTEEKPDGLNEATRQVALADLNKTDLVPEEDVKKLRTTRSNGLGQLETQRSRVDLSNVLDLHAFDSLSGSLQKKLQHVPGTQPHLDQSVTTFEVPGNAKEEHLNMFQNLLWEKNVRNKDNHCMEVRLKGLVSKDKSQQVVQGVHELYDLEETPVSWKDDTERTNRLVLGRNLDKDLKQLFATVTETEKQWTTHFKEDQVCT

SEQ ID NO: 13 - protein, 57 aa, Mus musculus
MAAVETRVCETDGCSSKLQCPTCIKLGIQGSYFCSQECFKGSWATHKLLHKKAKDEK

SEQ ID NO: 14 - protein, 59 aa, Homo sapiens
MAAVETRVCETDGCSSEAKLQCPTCIKLGIQGSYFCSQECFKGSWATHKLLHKKAKDEK

SEQ ID NO: 15 - protein, 59 aa, Danio rerio
MAAVETRECETEGCHSEAKLQCPTCIKLGIQGSYFCSQECFKGSWATHKLLHKKAKEDK

SEQ ID NO: 16 - protein, 59 aa, unidentified
  source 1..59: note = yeast
ASHPTKIYCSGLQCGRETSSQMKCPVCLKQGIVIFCDTSCYENNYKAHKALHNAKDGLE

SEQ ID NO: 17 - protein, 48 aa, Homo sapiens
SQTKKKQWCYNCEEEAMYHCCWNTSYCSIKCQQEHWHAEHKRTCRRKR

SEQ ID NO: 18 - protein, 52 aa, Homo sapiens
PNSDSSESCWNCGRKASETCSGCNTARYCGSFCQHKDWEKHHHICGQTLQAQ

SEQ ID NO: 19 - protein, 47 aa, Homo sapiens
GAMEQSCVNCGREAMSECTGCHKVNYCSTFCQRKDWKDHQHICGQSA

SEQ ID NO: 20 - DNA, 35 bp, Danio rerio
  source 1..35: mol_type = other DNA
gtggacccacagctcccggttaccattattactgg

SEQ ID NO: 21 - DNA, 29 bp, Danio rerio
  source 1..29: mol_type = other DNA
gtggacccacagctcaccattattactgg

SEQ ID NO: 22 - DNA, 40 bp, Danio rerio
  source 1..40: mol_type = other DNA
gtggacccacagctcagatcccggttaccattattactgg

SEQ ID NO: 23 - DNA, 11 bp, Danio rerio
  source 1..11: mol_type = other DNA
gctgcacagct

SEQ ID NO: 24 - DNA, 27 bp, Danio rerio
  source 1..27: mol_type = other DNA
gctgccacagcgaagccaagctcagtg

SEQ ID NO: 25 - DNA, 23 bp, Mus musculus
  source 1..23: mol_type = other DNA
tggagtcactggtgtccttaagg

SEQ ID NO: 26 - DNA, 34 bp, Mus musculus
  source 1..34: mol_type = other DNA
ttccagtcacaattgtcaccgggtacttaggtgc

SEQ ID NO: 27 - DNA, 32 bp, Mus musculus
  source 1..32: mol_type = other DNA
ttccagtcacaattcaccgggtacttaggtgc

SEQ ID NO: 28 - DNA, 32 bp, Mus musculus; identical to SEQ ID NO: 27
  source 1..32: mol_type = other DNA

SEQ ID NO: 29 - DNA, 27 bp, Mus musculus
  source 1..27: mol_type = other DNA
ttccagtcacaccgggtacttaggtgc

SEQ ID NO: 30 - DNA, 24 bp, synthetic construct
  misc_feature 1..24: note = primer1
  source 1..24: mol_type = other DNA
atggaggatgaggatgaatgtcca

SEQ ID NO: 31 - DNA, 26 bp, synthetic construct
  misc_feature 1..26: note = primer2
  source 1..26: mol_type = other DNA
ctactcgacgctgttatttgacacag

SEQ ID NO: 32 - DNA, 31 bp, synthetic construct
  misc_feature 1..31: note = primer3
  source 1..31: mol_type = other DNA
atgggatccatgttacctgctgtgaaaactg

SEQ ID NO: 33 - DNA, 29 bp, synthetic construct
  misc_feature 1..29: note = primer4
  source 1..29: mol_type = other DNA
atggaattcctagtgcgatggacaaactc

SEQ ID NO: 34 - DNA, 38 bp, synthetic construct
  misc_feature 1..38: note = primer5
  source 1..38: mol_type = other DNA
aagttctgttccaggggcccatggcggcggtggagacg

SEQ ID NO: 35 - DNA, 49 bp, synthetic construct
  misc_feature 1..49: note = primer6
  source 1..49: mol_type = other DNA
gagctcgaattcggggatccttaaaactgggacatgaaatgaggccgag

SEQ ID NO: 36 - DNA, 18 bp, synthetic construct
  misc_feature 1..18: note = primer7
  source 1..18: mol_type = other DNA
ggatccccgaattcgagc

SEQ ID NO: 37 - DNA, 18 bp, synthetic construct
  misc_feature 1..18: note = primer8
  source 1..18: mol_type = other DNA
gggcccctggaacagaac

SEQ ID NO: 38 - DNA, 26 bp, synthetic construct
  misc_feature 1..26: note = primer9
  source 1..26: mol_type = other DNA
atgggatccatggcggcggtggagac

SEQ ID NO: 39 - DNA, 32 bp, synthetic construct
  misc_feature 1..32: note = primer10
  source 1..32: mol_type = other DNA
atggaattctcagtaccgatagcctgcccatg

SEQ ID NO: 40 - DNA, 44 bp, synthetic construct
  misc_feature 1..44: note = primer11
  source 1..44: mol_type = other DNA
ctttaagaaggagatatacatatgttacctgctgtgaaaactgt

SEQ ID NO: 41 - DNA, 42 bp, synthetic construct
  misc_feature 1..42: note = primer12
  source 1..42: mol_type = other DNA
tcgacgtaggcctttgaattcctagtgcgatggacaaactcc

SEQ ID NO: 42 - DNA, 38 bp, synthetic construct
  misc_feature 1..38: note = primer13
  source 1..38: mol_type = other DNA
gaacagattggaggatcccatatggcggcggtggagac

SEQ ID NO: 43 - DNA, 44 bp, synthetic construct
  misc_feature 1..44: note = primer14
  source 1..44: mol_type = other DNA
tcgagtgcggccgcaagcttttaaaactgggacatgaaatgagg

SEQ ID NO: 44 - DNA, 50 bp, synthetic construct
  misc_feature 1..50: note = primer15
  source 1..50: mol_type = other DNA
tcgagtgcggccgcaagcttttacttttcatcttttgctttcttatgtag

SEQ ID NO: 45 - DNA, 38 bp, synthetic construct
  misc_feature 1..38: note = primer16
  source 1..38: mol_type = other DNA
tcgagtgcggccgcaagcttttatgcccatgggtcagt

SEQ ID NO: 46 - DNA, 60 bp, synthetic construct
  misc_feature 1..60: note = primer17
  source 1..60: mol_type = other DNA
ccaattcaggacaatcctccgcgtactcttcttccgctcctccaatctgttcgcggtgag

SEQ ID NO: 47 - DNA, 60 bp, synthetic construct
  misc_feature 1..60: note = primer18
  source 1..60: mol_type = other DNA
gaggattgtcctgaattggtccccattgagaccaagaaccaagaaatggcggcggtggag

SEQ ID NO: 48 - DNA, 40 bp, synthetic construct
  misc_feature 1..40: note = primer19
  source 1..40: mol_type = other DNA
aagttctgttccaggggcccatggaggatgaggatgaatg

SEQ ID NO: 49 - DNA, 43 bp, synthetic construct
  misc_feature 1..43: note = primer20
  source 1..43: mol_type = other DNA
gagctcgaattcggggatccctactcgacgctgtctttatttg

SEQ ID NO: 50 - DNA, 44 bp, synthetic construct
  misc_feature 1..44: note = primer21
  source 1..44: mol_type = other DNA
aagttctgttccaggggcccatgactgcagcagcagacagcagc

SEQ ID NO: 51 - DNA, 43 bp, synthetic construct
  misc_feature 1..43: note = primer22
  source 1..43: mol_type = other DNA
gagctcgaattcggggatccctacatttgggacaggaagtgcg

SEQ ID NO: 52 - DNA, 26 bp, synthetic construct
  misc_feature 1..26: note = primer23
  source 1..26: mol_type = other DNA
aattaatacgactagctagaaatagc

SEQ ID NO: 53 - DNA, 80 bp, synthetic construct
  misc_feature 1..80: note = primer24
  source 1..80: mol_type = other DNA
ttttgcaccgactcggtgccactttttcaagttgataacggactagccttattttaacttgctatttctagctctaaaac

SEQ ID NO: 54 - DNA, 60 bp, synthetic construct
  misc_feature 1..60: note = primer25
  source 1..60: mol_type = other DNA
aattaatacgactcactataggaggtgggacactggagctgttttagagctagaaatagc

SEQ ID NO: 55 - DNA, 26 bp, synthetic construct
  misc_feature 1..26: note = primer26
  source 1..26: mol_type = other DNA
ctgcctgttaatagttaatcagacag

SEQ ID NO: 56 - DNA, 26 bp, synthetic construct
  misc_feature 1..26: note = primer27
  source 1..26: mol_type = other DNA
actgactgattacttcaagcaatctg

SEQ ID NO: 57 - DNA, 25 bp, synthetic construct
  misc_feature 1..25: note = primer28
  source 1..25: mol_type = other DNA
ctctgatctcagatgactgcagcag

SEQ ID NO: 58 - DNA, 24 bp, synthetic construct
  misc_feature 1..24: note = primer29
  source 1..24: mol_type = other DNA
ctgagagcaaaagtatgagccttg

SEQ ID NO: 59 - DNA, 26 bp, synthetic construct
  misc_feature 1..26: note = primer30
  source 1..26: mol_type = other DNA
cgggaaggcgtgtctagtttgaatag

SEQ ID NO: 60 - DNA, 28 bp, synthetic construct
  misc_feature 1..28: note = primer31
  source 1..28: mol_type = other DNA
ctctaaactgaggggagatgagcttcac

SEQ ID NO: 61 - DNA, 20 bp, synthetic construct
  misc_feature 1..20: note = primer32
  source 1..20: mol_type = other DNA
gcgccgactgaatcctgtcg

SEQ ID NO: 62 - DNA, 21 bp, synthetic construct
  misc_feature 1..21: note = primer33
  source 1..21: mol_type = other DNA
ctcgaagattcgtcctcggcc

SEQ ID NO: 63 - DNA, 19 bp, synthetic construct
  misc_feature 1..19: note = primer34
  source 1..19: mol_type = other DNA
cttctcaggctgactgtgc

SEQ ID NO: 64 - DNA, 18 bp, synthetic construct
  misc_feature 1..18: note = primer35
  source 1..18: mol_type = other DNA
ccgctaggattaccctcc

SEQ ID NO: 65 - DNA, 24 bp, synthetic construct
  misc_feature 1..24: note = primer36
  source 1..24: mol_type = other DNA
cacttgtccctctaagaagttgca

SEQ ID NO: 66 - DNA, 22 bp, synthetic construct
  misc_feature 1..22: note = primer37
  source 1..22: mol_type = other DNA
ggttgattccgataacgaacga

SEQ ID NO: 67 - DNA, 23 bp, synthetic construct
  misc_feature 1..23: note = primer38
  source 1..23: mol_type = other DNA
ggatacttgggtgcaggaaagac

SEQ ID NO: 68 - DNA, 21 bp, synthetic construct
  misc_feature 1..21: note = primer39
  source 1..21: mol_type = other DNA
ctctcctgcctgactcacagc

SEQ ID NO: 69 - DNA, 18 bp, synthetic construct
  misc_feature 1..18: note = primer40
  source 1..18: mol_type = other DNA
ggctggaacttagaaatg

SEQ ID NO: 70 - protein, 59 aa, synthetic construct; identical to SEQ ID NO: 14
  REGION 1..59: note = predicted

(19) United States
(12) Patent Application Publication
(10) Pub. No.: US 2025/0304625 A1
(43) Pub. Date: Oct. 2, 2025

(54) ARTICLES AND METHODS FOR INHIBITING METHIONINE AMINOPEPTIDASE ACTIVITY

(71) Applicant: Vanderbilt University, Nashville, TN (US)

(72) Inventors: Eric P Skaar, Nashville, TN (US); Caitlin Murdoch, Nashville, TN (US); Andy Weiss, Nashville, TN (US)

(21) Appl. No.: 18/851,638

(22) PCT Filed: Apr. 6, 2023
(86) PCT No.: PCT/US2023/017796

§ 371 Date: Sep. 26, 2024

Related U.S. Application Data
(60) Provisional application No. 63/328,213, filed on Apr. 6, 2022.

(51) Int. Cl. C07K 14/00; A61K 38/00; A61K 45/06; A61P 35/00

(52) U.S. Cl. CPC C07K 14/001; A61K 45/06; A61P 35/00; A61K 38/00

(57) ABSTRACT

A method of treating a disease or condition characterized by increased activity of a methionine aminopeptidase (METAP) protein includes interfering with a transfer of Zn to the METAP protein from a Zn-regulated GPTase metalloprotein activator 1 (ZNG1) protein. A method of reducing an activity of a methionine aminopeptidase (METAP) protein is also provided, including interfering with a transfer of Zn to the METAP protein from a Zn-regulated GPTase metalloprotein activator 1 (ZNG1) protein

### RELATED APPLICATION

This application is a § 371 National State Application of PCT/US2023/017796 filed Apr. 6, 2023, which claims priority from U.S. Provisional Application Ser. No. 63/328,213 filed Apr. 6, 2022, the entire disclosure of which is incorporated herein by this reference.

### GOVERNMENT INTEREST

This invention was made with government support under grant numbers P30CA068485 and R01AI150701, awarded by the National Institutes of Health (NIH). The government has certain rights in the invention.

### REFERENCE TO AN ELECTRONIC SEQUENCE LISTING

The contents of the electronic sequence listing (Skaar-11672N-22117US.xml; Size: 81,466 bytes; and Date of Creation: Apr. 29, 2025) is herein incorporated by reference in its entirety.

### TECHNICAL FIELD

The present-disclosed subject matter relates to articles and methods for inhibiting methionine aminopeptidase activity. In particular, the presently-disclosed subject matter relates to inhibition of methionine aminopeptidase activity through targeting of ZNG1 or inhibition of metalation of methionine aminopeptidase by ZNG1 for the treatment of diseases and conditions such as obesity and cancer.

### BACKGROUND

Metals are essential micronutrients that are indispensable for cellular processes in all kingdoms of life. Zinc (Zn) is the second most abundant transition metal in humans serving as a structural or enzymatic cofactor for approximately 10% of the proteome (Andreini et al., 2006). Consequently, perturbations in Zn homeostasis are linked to human disease, including growth deficiencies, immune defects, neurological disorders, and cancers (Basu, 2018; Devirgiliis et al., 2007; Fischer Walker and Black, 2004; Prasad, 2013). This is particularly alarming as Zn deficiency is the fifth most important risk factor for mortality in developing countries affecting close to half of the world's population (Guilbert, 2003).

Members of the ubiquitous G3E family of P-loop GTPases deliver/insert different metal cofactors to client metalloproteins (Haas et al., 2009). This family includes COG0523 proteins, a subgroup found across all branches of life with poorly understood cellular functions (Edmonds et al., 2021; Haas et al., 2009). COG0523 proteins are characterized by a conserved N-terminal GTPase domain and GTP hydrolysis is thought to provide energy for the transfer of metals to client proteins (Jordan et al., 2019). Furthermore, COG0523 proteins have been implicated in cellular Zn homeostasis as the expression of several bacterial and eukaryotic COG0523 members is induced during conditions of Zn starvation (Coneyworth et al., 2012; Haas et al., 2009; Jordan et al., 2019; Mortensen et al., 2014; Ogo et al., 2015). Despite the assignment of COG0523 proteins as putative nucleotide hydrolysis-powered Zn metallochaperones that act under conditions of Zn restriction, conclusive experimental evidence in support of this proposed function has not been provided.

Cellular Zn is present at levels similar to major metabolites like ATP; however, the majority of Zn within the cell is associated with Zn-requiring metalloproteins, Zn-storage proteins, or maintained in vesicular storage (Krezel and Maret, 2017; Sigel et al., 2013; Wellenreuther et al., 2009). This tight regulation of intracellular pools results in extremely low levels of freely available Zn (Ba et al., 2009). During Zn limitation, the metalation of critical metalloproteins is thought to require the hierarchical distribution of Zn to ensure function of these proteins. Akin to other metals like copper (O'Halloran and Culotta, 2000), targeted transfer of Zn to metalloproteins is hypothesized to be mediated by specialized proteins referred to as metallochaperones/metal-insertases (Rosenzweig, 2002), yet no such protein has been identified to date.

Many proteins require metal co-factors (e.g., zinc) for proper function. One group of zinc-dependent proteins are methionine aminopeptidases (METAPs), which process newly synthesized proteins to ensure their proper function. Due to their importance for de novo protein synthesis and proteostasis, METAPs are uniformly expressed across the human body, and thought to be particularly important for proliferating cells. One hallmark of cancer is accelerated cellular proliferation and increased expression of human METAP2 has been implicated in tumorigenesis. This connection has led to the development of METAP2-inhibitors including TNP-470 as pharmacological interventions to treat various cancers (e.g., prostate).

While generally promising, such treatments are hampered by dose-limiting toxic side effects, therefore restricting their therapeutic potential. Of note, although the role of METAP2 for oncogenesis has been well established, little is known about the influence of METAP1 on cancer progression. This is particularly important, as METAP1 could aid in overcoming the effects of METAP2 inhibitors through the redundancy between the METAP proteins. Additionally, METAP enzymes have been linked to other non-communicable diseases including obesity. Known METAP2 inhibitors have been shown to elicit anti-obesogenic activity via inhibition of the sterol regulatory element binding protein (SREB), a factor involved in lipid and cholesterol biosynthesis, thereby placing drugs that target METAP activity as therapies for metabolic syndromes.

The present inventors have recently identified a novel vertebrate family of proteins that distribute Zn within the cell, which they have named Zn regulated GTPase metalloprotein activator 1 (ZNG1) family. The present disclosure shows that ZNG1 functions as a metallochaperone to deliver zinc to METAP1, therefore supporting its function. Consequently, targeted inhibition of ZNG1 (e.g., in cancer cells) or interference with ZNG1/METAP1 interaction is believed to decrease METAP1 activity. Such targeted inhibition/interference shows promise as a treatment for cancer and obesity either alone or in conjunction with known METAP2 inhibitors to circumvent compensatory effects by METAP1.

### SUMMARY

In accordance with the purposes and benefits described herein, novel methods for treatment of diseases and/or conditions facilitated by cells dependent on metallation are described. In one aspect, a method for treating a disease or condition such as obesity or cancer is described, comprising inhibiting methionine aminopeptidase (METAP) activity. In some embodiments, the method includes inhibiting METAP1 activity. In some embodiments the method includes targeting Zn regulated GTPase metalloprotein activator 1 (ZNG1) family proteins and/or the interaction of METAP and ZNG1 proteins.

In one aspect of the disclosure, a method of treating a disease or condition characterized by increased activity of a methionine aminopeptidase (METAP) protein is disclosed, comprising interfering with a transfer of Zn to the METAP protein from a Zn-regulated GPTase metalloprotein activator 1 (ZNG1) protein. The disease or condition may be characterized by uncontrolled cellular proliferation or may be a metabolic disease or condition. In embodiments, the disease or condition is obesity or a cancer. The METAP protein may be METAP1. The method may optionally include administering one or more METAP2 inhibitors.

In embodiments, the interfering may comprise reducing or preventing the transfer of Zn from the ZNG1 protein to the METAP protein. In other embodiments, the interfering may comprise reducing or inhibiting an activity of the ZNG1 protein. In embodiments, the reducing or preventing the transfer of Zn comprises reducing or preventing an interaction between the ZNG1 protein and the METAP protein. In one possible embodiment, the reducing or preventing an interaction is effected by a peptide comprising the amino acid sequence:

(SEQ ID NO:_ 70)
MAAVETR VCETDGCSSEAKLQCPTCIKLGIQGSYFCSQECFKG
SWATHKLLHKKAKDEK.

In another aspect of the disclosure, a method of reducing an activity of a methionine aminopeptidase (METAP) protein, comprising interfering with a transfer of Zn to the METAP protein from a Zn-regulated GPTase metalloprotein activator 1 (ZNG1) protein. The METAP protein may be METAP1.

In embodiments, the interfering may comprise reducing or preventing the transfer of Zn from the ZNG1 protein to the METAP protein. In other embodiments, the interfering may comprise reducing or inhibiting an activity of the ZNG1 protein. In embodiments, the reducing or preventing the transfer of Zn comprises reducing or preventing an interaction between the ZNG1 protein and the METAP protein. In one possible embodiment, the reducing or preventing an interaction is effected by a peptide comprising the amino acid sequence:

(SEQ ID NO:_ 70)
MAAVETRVCETDGCSSEAKLQCPTCIKLGIQGSYFCSQECFKGS
WATHKLLHKKAKDEK.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Identification of metalloprotein targets of Zn regulated GTPase metalloprotein activator 1 (ZNG1) in vertebrates. (A) Cladogram and analysis of ZNG1 protein sequence conservation in vertebrates over evolutionary time. Numbers on branches denote nucleotide substitutions per site. (B) Amino acid conservation along the length of vertebrate ZNG1s indicates high sequence conservation particularly in the GTPase motifs and at the N-terminus. Lighter shading reflects higher conservation. (C) Yeast-two-hybrid screens using full-length human, mouse, and zebrafish ZNG1s identify several unique and shared interacting proteins. (D) Molecular function enrichment analysis of ZNG1 interaction protein PFAM domains. (E) Number and cellular activity of ZNG1 client Zn metalloproteins detected in yeast-two-hybrid screens. (F) ZNG1 interaction domains in Zn metalloproteins shown in E. (G) Conserved ZNG1 interaction domain of METAP1 across species overlaps with a zf-C6H2 domain. Yellow shading indicates minimal conserved region across species.

FIG. 2. ZNG1 interacts with METAP1 via a unique C6H2 Zn finger domain. (A) Affinity chromatography of mouse (Mm) His-MBP-ZNG1 immobilized on amylose resin, with flow-through (FT) and washes (W1-3) prior to addition of free Mm METAP1, with subsequent FT and W4-6 prior to elution (E) of His-MBP-ZNG1, and detection by SDS PAGE (top 2 panels) and by immunoblot (bottom). (B) DLS analysis of full-length Mm ZNG1 and METAP1 shows complex formation in vitro. (C) Size exclusion chromatography of full-length Mm ZNG1 and METAP1₁₋₈₆. (D) Identification of a conserved N-terminal ‘CPELVPI’ motif in Mm, human (Hs), and zebrafish (Dr) ZNG1s and synthetic peptides. (E) Spectra of W45 fluorescence quenching of 3 μM METAP1₋₅₉ upon Mm ZNG1 N-terminus binding. (F) Peptide binding curve from (E) fit to a 1:1 binding model. (G) Measured affinities of METAP1₁₋₅₉ and METAP1₋₇₉ binding to Mm, Hs, and Dr ZNG1 N-termini. (H) ¹H ¹⁵N HSQC NMR spectra of METAP1₁₋₅₉ alone (left), in complex with Mm ZNG1 N-terminal peptide (middle), and as N-terminal Mm ZNG1 peptide fusion (right). (I) Chemical shift perturbations in ¹⁵N METAP1₁₋₅₉ upon binding to Mm ZNG1 peptide.

FIG. 3. NMR structure of METAP1-ZNG1 complex. (A) ¹H, ¹⁵N NOE for the fusion (black) and free domain (red). (B) Structure of the 20 lowest-energy conformers of the full fusion construct, colored by ¹H, ¹⁵N NOE. (C) Ribbon diagram of the Mm ZNG1 10-30 METAP1₋₅₉ fusion showing Zn coordination. (D) Stereo view of 20 lowest-energy conformers of the Mm ZNG1₁₀₋₃₀ METAP1₁₋₅₉ fusion, colored by secondary structure, with helices in green and beta strands in purple, and ZNG1 10-30 in blue. (E) Electrostatic surface map of the METAP11-59 peptide interface. (F) N-terminal ZNG1 motif among eukaryotic cluster 3 COG0523 proteins.

FIG. 4. ZNG1 enhances METAP1 aminopeptidase activity in vitro. (A) Representative titration of 1.0 μM METAP1, 1.0 μM quin-2 with Zn. (B) Representative titration of 1.3 μM ZNG1, 1.3 μM quin-2 with Zn. Solid lines show a global fit of independent replicates; dashed lines are simulations of 10-fold greater and weaker affinities. (C) GTPase activity of ZNG1 in the presence or absence of Zn and/or Zn₂METAP1. (D) Initial rate of Met release from 750 μM MAHAIHY peptide by 20 nM METAP1 in different metalation states. (E) Activation of 10 μM Zn₂METAP1 enzymatic activity by 25 μM ZNG1 in the presence or absence of Zn and 500 μM guanine nucleotides. (F) Activation of 10 μM Zn₂METAP1 enzymatic activity by 22.5 μM Zn or 25 μM ZnZNG1 in the presence or absence of 500 μM guanine nucleotides, all in the presence of 250 μM NTA. METAP1 reactions in panels E and F were conducted by diluting activation reactions to 20 nM METAP1 and monitoring cleavage of 750 μM MAHAIHY. (G) Mechanistic model of GTP hydrolysis-coupled Zn transfer from ZNG1 to the METAP1 active site.

FIG. 5. zng1 mutant zebrafish display Zn-dependent sensitivity to Metap inhibition and phenocopy metap1 mutant larvae. (A) zng1 expression levels following TPEN treatment in WT and zng1^{-/-} mutant 6 dpf whole zebrafish larvae. (n=4 pools of 30 larvae per genotype/treatment group). (B-D) Measurement of height at anterior anal fin (HAA) (B), standard length (C), and snout to vent length (D) of zng1^{-/-} and WT 6 dpf larvae. Each data point represents an individual larva. (E) Survival of WT and zng1^{-/-} mutant larvae treated with Bengamide B and TPEN (n=3 groups of 5 larvae/genotype/treatment) over 3 days of treatment and terminating at 6 dpf. (F) Brightfield images of 6 dpf metap1 mutant larvae treated with 1 μM Bengamide B or vehicle control. Yellow arrows denote abnormal pathologies. Pericardial Edema (PCE), craniofacial defects (CD), yolk sack edema (YSE), and uninflated swim bladder (USB). Scale bars=1 mm. (G) Quantification of gross pathology score of 6 dpf Bengamide B treated metap1 mutant and WT larvae. Pathology scores calculated by taking the sum of each pathology indicated by the yellow arrows in panel F. Each data point represents an individual larva. (H) Brightfield images of 6 dpf zng1 mutant larvae treated with 500 nM Bengamide B or vehicle control. Scale bars=1 mm. (I) Quantification of gross pathology score of 6 dpf Bengamide B treated zng1 mutant larvae as depicted above. (J) Brightfield images of 6 dpf WT larvae treated with 1 μM Bengamide B or vehicle control. Scale bars=1 mm. (K) Survival of WT and zng1^{-/-} larvae treated with TNP-470 and TPEN (n=3 groups of 5 larvae/genotype/treatment) over 3 days of treatment and terminating at 6 dpf. Data in panels A, E, and K analyzed by two-way ANOVA with Tukey's multiple comparison test. Data in panels B,C,D, G and I analyzed by Student's t-test.

FIG. 6. Zng1 mutant mice exhibit signatures of mitochondrial dysfunction on a Zn deficient diet. (A) Genotype distribution of WT, Zng1^{+/-}, and Zng1^{-/-} mice. Number of animals per genotype indicated in white text. (B) Percent weight gain of 5-7 week old WT and Zng1 mutant mice that were placed on a Zn-deficient diet for 6 weeks (n=4-5 mice/genotype/sex). (C) Differential protein abundances from WT and Zng mutant dissected kidneys from 11-13 week old female mice that were maintained on a low Zn diet for 5 weeks. Proteins that localize to the mitochondria are highlighted in red (n=5 mice/genotype). (D) IPA analysis of differentially abundant proteins (Zng1^{-/-}/WT) with significance of gene enrichment and number of differentially abundant proteins for each pathway depicted. z-scores are denoted within teal bars and indicate the predicted effects on each pathway (positive values: activation; negative values: inhibition, n.a.: no prediction through IPA available due to insufficient evidence in the Knowledge Base). Red bars indicate enriched proteins in Zng1 mutant animals and blue bars represent proteins with reduced abundance. Data in panel A analyzed by Chi-squared goodness of fit test. Data in panel B analyzed by two-way ANOVA with Sidak's multiple comparison test within each sex.

FIG. 7. ZNG1 regulates cellular respiration through stimulation of METAP activity. (A) Expression of Zng1 in TKPTS cells treated with TPA. (B) Zng1 transcript levels in untreated WT and Zng1 mutant TKPTS cells. (C-D) Proliferation of TKPTS cells quantified by CellTrace median fluorescence intensity (MFI) in Zn-deplete conditions (C) and following treatment with METAP2 inhibitor TNP-470 (D) using flow cytometry. (E) Ratio of processed 14-3-3γ protein levels in vehicle or TPA treated Zng1^{-/-}/WT TKPTS cells. (n=4/genotype/treatment). (F) Representative TEM images of WT and Zng1 mutant TKPTS cells grown in Zn-deplete conditions (scale bars=500 nm). Red box (top panel) outlines region of higher magnification (bottom panel). (G-H) Non-linear regression analysis of the size distribution of mitochondria (G) and mean signal intensity (H) in WT and Zng1 mutant TKPTS cells treated with TPA. (WT n=112 mitochondria, Zng1^{-/-} n=101 mitochondria). (I) ATP levels in cell lysates from untreated WT and Zng1 mutant TKPTS cells. (J) Mitochondrial membrane potential (ΔΨₘ) in untreated and TPA treated TKPTS cells. (K) Production of mitochondrial superoxide in TKPTS cells treated with TNP-470 was quantified by flow cytometry. (L-M) Oxygen consumption rates (OCR) by TKPTS cells treated with vehicle or TPA. OCR was normalized by cell number. Data in panels A, B, H, and I analyzed by Student's t-test. Data in panel E analyzed by Welch's t-test. Data in panel G analyzed by non-linear regression goodness of fit. Data in panels C, D, J, K, and M analyzed by two-way ANOVA with Tukey's multiple comparison test.

FIG. 8. Conservation and tissue specific expression of Human ZNG1s. (A) Multiple sequence alignment of vertebrate ZNG1 proteins depicted in cladogram of FIG. 1 panel A. White shading in the bottom bar indicates high conservation. (B) Expression levels of human ZNG1A, ZNG1B, ZNG1C, ZNG1E, and ZNG1F across different tissue and cell types. Expression data obtained from the human protein atlas (Uhlen et al., 2015). (C) Multiple sequence alignment of human ZNG1 paralogs and consensus sequence.

FIG. 9. Biochemical interaction of ZNG1 and METAP1. (A-C) Affinity chromatography of full-length immobilized zebrafish MBP-Metap1 bound amylose (A) or empty control resin (B) with purified zebrafish Zng1 passed over (FT, flow through). Columns were serially washed (W). Elution of Metap1 (denoted E) resulted in detection of Zng1 shown on SDS PAGE (top two panels) and by immunoblot (bottom panel). Immunoblot signal quantified by densitometry (C). (D) Size exclusion chromatography of mouse METAP₁₋₈₆ interaction with full-length ZNG1 visualized by SDS PAGE. (E) Alignment of METAP1 C2H6 zf domains from zebrafish (Dr), mouse (Mm), human (Hs), and Saccharomyces cerevisiae (Sc) with MYND zf domains from indicated Hs proteins AML-1, BS69, and DEAF-1. (F) Ribbon diagrams of previously determined structures of zf-MYND: peptide complexes (Harter et al., 2016; Liu et al., 2007). (G) Consensus sequence logo of zf-MYND and zf-C6H2 domains. (H) Metal stoichiometries of Mm METAP1₁₋₅₉ and METAP1₁₋₇₉ as purified and after loading with Zn as measured by ICP-MS. ‘n.d.’ (not detected) indicates measured metal concentrations below 0.01 equivalents. (I) Representative spectra of fluorescence quenching of W45 in Mm METAP1₁₋₇₉ by titration with Mm ZNG1 peptide. (J) ¹H ¹⁵N HSQC NMR spectrum of Mm METAP1₁₋₅₉ in 2:1 complex with Mm ZNG1 N-terminal peptide, showing equal populations in bound and free states. Data in panel C are represented as mean±SEM.

FIG. 10. NMR structural characterization of ZNG1 and METAP1. (A) Assigned ¹H ¹⁵N HSQC NMR spectra of Mm METAP1₁₋₅₉ alone (left), in 1:1 complex with Mm ZNG1 N-terminal peptide (middle), and with N-terminally fused Mm ZNG1 N-terminal peptide (right). (B) Chemical shift-based secondary structure predictions for Mm METAP1₁₋₅₉ alone (top), the 1:1 complex with Mm ZNG1 N-terminal peptide (middle), and with N-terminally fused Mm ZNG1 N-terminal peptide (bottom). (C) Sequence diagram of fusion construct of Mm ZNG1 N-terminal peptide with Mm METAP1₁₋₅₉. (D) Overlay of ¹H ¹⁵N HSQC NMR spectra of Mm METAP1₁₋₅₉ in 1:1 complex with Mm ZNG1 N-terminal peptide (blue contours) and with N-terminally fused Mm ZNG1 N-terminal peptide (red contours), showing the fusion strategy eliminates peak splitting artifacts. (E) Chemical shift perturbations caused by the fusion construct, relative to the complex. (F) ¹⁵N R1 relaxation rates for the fusion (black) and free domain (red). (G) ¹⁵N R2 relaxation rates for the fusion (black) and free domain (red). (H) Surface representation of the fusion structure, colored by ¹H, ¹⁵N chemical shift perturbations upon peptide binding. The peptide is shown as blue sticks. P23 is colored black. (I) Ribbon diagram and electrostatic surface representation showing the predicted peptide binding interface of the S. cerevisiae Mapp zf-C6H2 domain with a Zng1p peptide.

FIG. 11. In vitro METAP1 activity assay. (A) Unfolding of METAP1₁₋₅₉ as measured by a decrease in W45 fluorescence as a function of EDTA concentration and time. (B) Unfolding of METAP1₁₋₅₉ as monitored by ¹⁵N-HSQC spectra recorded at increasing concentrations of EDTA. METAP1₁₋₅₉ in the presence of 50 mM EDTA gives spectra indicative of protein aggregation. (C) Metal stoichiometries of Mm METAP1 as purified, supplemented with excess Zn, or when treated with various metal chelators as measured by ICP-MS. ‘n.d.’ (not detected) indicates measured metal concentrations below 0.01 equivalents. (D) Zn₂METAP1 Zn binding. Representative titration of Zn into 1 μM Zn₂METAP1 and 0.8 μM mf2. (E) ZNG1 Zn binding. Representative titration of Zn into 7.5 M ZNG1 and 16 μM mf2. For panels D and E, solid lines represent the global fit from three independent replicates and dashed lines represent simulations of 10-fold greater and weaker affinities. (F) Zn binding affinities of Mm METAP1 and Mm ZNG1 as measured by competition with quin2 and mf2. (G) ZNG1 GTPase activity as measured by enzyme-coupled assay in Apo and Zn states. (H) Representative time-course analysis of the cleavage of 125 μM MAHAIHY peptide by Zn₄METAP1 by monitoring the release of Met. Indicated timepoint was analyzed by LC-ESI-MS (see panel I). (I) Extracted ion chromatograms of product AHAIHY (top) and substrate MAHAIHY (bottom) peptides taken from the indicated 10 min reaction from panel H. (J) Initial rates of MAHAIHY peptide cleavage at varying substrate concentrations by Zn₄METAP1. (K) Michaelis-Menten kinetics of the Zn₄METAP1 cleavage of the MAHAIHY peptide. (L) Initial rate of cleavage of 750 μM MAHAIHY by 20 nM murine Zn₂METAP1 or Zn₄METAP1 in the presence or absence of Apo or Zn-bound murine ZNG1 in the absence of nucleotide. (M) Cleavage of 100 μM MAHAIHY peptide after incubation of 1 μM Dr Zng1 and 1 μM Metap1. Data are represented as mean±SEM. Data in panels M analyzed by one-way ANOVA with Tukey's multiple comparison test. Significance denoted as: * P<0.05, ** P<0.01, *** P<0.001, **** P<0.0001.

FIG. 12. Generation of zng1 and metap1 mutant zebrafish. (A) Schematic of zebrafish zng1 locus with gRNA shown by red bar in second exon. Mutant alleles and in silico translations indicated. Arrows indicate screening primer binding sites. (B) Genotype distributions of WT, zng1−/+, and homozygous zng1^{-/-} 6 dpf larvae. Number of animals indicated in white text. (C) Survival of WT and zng1^{-/-}mutant larvae treated with Bengamide B and TPEN (n=3 groups of 5 larvae/genotype/treatment) over the course of 3 days of treatment. (D) Measurement of height at anal fin (HAA) of WT 6 dpf larvae following three days of exposure to 500 nM Bengamide B or vehicle control. Each data point represents an individual larva. (E) Schematic of zebrafish metap1 locus with gRNA shown by red bar in first exon. Mutant allele and in silico translations indicated. Arrows indicate screening primer binding sites. (F) Genotype distributions of WT, metap1^{-/+}, and homozygous metap1^{-/-} 6 dpf larvae. Number of animals indicated in white text. (G) Percent of WT and metap1^{-/-}-6 dpf larvae with pericardial edema, uninflated swim bladder, craniofacial abnormalities, or yolk sac edema following treatment with 1 μM Bengamide B or vehicle control for 3 days. (n=6-8 larvae/genotype). (H) Percent of WT and zng1-6 dpf larvae with pericardial edema, uninflated swim bladder, craniofacial abnormalities, or yolk sac edema following treatment with 500 nM Bengamide B of vehicle control for 3 days. (n=18-20 larvae/genotype). (I) Survival of WT and zng1^{-/-} larvae treated with TNP-470 and TPEN (n=3 groups of 5 larvae/genotype/treatment) over the course of 3 days of treatment. Data are represented as mean±SEM. Data in panels B and F analyzed by Chi-square goodness of fit test. Data in panel D analyzed by Student's t-test. Significance denoted as: * P<0.05, ** P<0.01, *** P<0.001, **** P<0.0001.

FIG. 13. Generation of Zng1 mutant mice. (A) Schematic of mouse Zng1 locus with gRNA shown by red bar in first exon. (B) RT-qPCR of Zng1 transcript in WT and Zng1 mutant kidneys (n=4 mice/genotype). Normalized to Actb expression. (C) Normalized peak area of ZNG1 peptides in WT and Zng1^{-/-}tissue (brain) measured using data-independent acquisition mass spectrometry proteomics. (D-E) Breeding patterns of male and female mice resulting from Zng1 heterozygous crosses. Number of animals indicated in white text. (F) 5-7 week old male or female mice were placed on a control diet for the course of 6 weeks and percent weight gain measured (n=5-8 mice/genotype/sex). (G) 5-7 week old female mice were placed on a control or low Zn diet for the course of 6 weeks and tissue Zn levels assessed (n=6-8 mice/genotype/sex). (H) Summary network of ATPIF1 upstream regulatory connections identified by IPA. Analysis was based on all differentially abundant proteins (P-value≤0.05) from proteomics comparing renal protein content from WT and Zng1 mutant mice (FIG. 6C). (I) RT-qPCR analysis of transcripts encoding for most differentially abundant proteins in Zng1 mutant mice (FIG. 6C, Table S3). Normalized to expression of Ppia, Pgam, and 18S genes. For panels A, B and I, protein or RNA was isolated from tissue from 11-13 week old female mice that were maintained on a low Zn diet for 5 weeks. Data are represented as mean±SEM. Data in panels B, C, and I analyzed by Student's t-test. Data in panels D and E analyzed by Chi-square goodness of fit test. Data in panel F and G analyzed by two-way ANOVA with Sidak's post-test. In panel F two-way ANOVA was run for each sex independently. Significance denoted as: * P<0.05, ** P<0.01, *** P<0.001, **** P<0.0001.

FIG. 14. Generation and validation of Zng1 mutant alleles in TKPTS cells. (A) Sequence alignment of WT and Zng1 mutant alleles generated by CRISPR/Cas9. Two independent mutant clones were isolated, clone 1 (Δ2/Δ2) and clone 2 (42/47) with deletion alleles that result in frameshift mutations. (B) Immunoblot for METAP1 in WT and Zng1^{-/-} TKPTS cells. (C) Immunoblot for ZNG1 and Tubulin control in WT, Zng1^{-/-}, and Zng1^{-/-}+Zng1 TKPTS cells. (D) Zng1 transcript levels in WT, Zng1 clone 1, and Zng1 clone 2 TKPTS cells. (E-F) CellTrace fluorescence levels in WT, Zng1 clone 1, and Zng1 clone 2 TKPTS cells treated with TPA (E). FACS histogram associated with quantification of indicated panel in FIG. 7 (F). (G) Proliferation measured by CellTrace fluorescence of untreated and Bengamide B treated WT TKPTS cells. (H-I) CellTrace fluorescence levels in WT, Zng1 clone 1, and Zng1 clone 2 TKPTS cells treated with TNP-470 (H). Histogram associated with quantification of indicated panel in FIG. 7 (I). (J) Proliferation measured by CellTrace fluorescence of untreated and TNP-470 treated WT, Zng1^{-/-}, and Zng1^{-/-}+Zng1 TKPTS cells. (K-L) Proteomics was performed on WT and Zng1^{-/-} TKPTS cells grown in the presence of TPA or Bengamide B. The abundance of iMet-containing and processed N-termini of 14-3-3γ (MVDREQLVQK and VDREQLVQK, respectively) in WT and mutant cells upon TPA treatment was determined to assess the % of unprocessed protein (K). Changes to canonical pathways (as determined by IPA) comparing protein signatures from TKPTS cells with impaired Zng1 and WT cells upon inhibition of METAP with Bengamide B were determined (L). Depicted are pathways that meet the following criteria for one of the two conditions: significance cutoff of P-value (−log 10)≥1.3, |z-score|≥2. Pathways with the highest total score (combined p-value and z-score) are on top, and color-coding reflects z-score (positive values: activation; negative values: inhibition). Grey boxes denote pathways for which no z-score was determined due to a limited number (<4) proteins affected in a specific pathway. (M) Mitochondrial roundness of WT and Zng1 mutant TKPTS cells grown in Zn-deplete conditions as quantified from TEM. (N) TMRM fluorescence in WT, Zng1^{-/-} clone 1, and Zng1^{-/-} clone 2 TKPTS cells. (O) MitoTracker levels WT, Zng1 clone 1, and Zng1 clone 2 TKPTS cells. (P-Q) MitoSOX levels WT, Zng1^{-/-}clone 1, and Zng1^{-/-} clone 2 TKPTS cells treated with TNP-470. (N) FACS histogram associated with quantification of indicated panel in FIG. 7. (R) Mitochondrial superoxide measured by MitoSOX levels in untreated and TNP-470 treated WT, Zng1^{-/-}, and Zng1^{-/-} +Zng1 TKPTS cells. (S) Confocal micrographs of WT and Zng1^{-/-} cells illustrating ATPIF1 distribution (magenta), mitochondria (cyan), and DAPI (grey). Scale bar=20 μm Data are represented as mean±SEM. Data in panels D and N were analyzed by one-way ANOVA. Data in panels K and M analyzed by Student's t-test. Data in panels E,G,H,J,O,Q and R were analyzed by two-way ANOVA with Tukey's multiple comparison test. Significance denoted as: * P<0.05, ** P<0.01, *** P<0.001, P<0.0001.

FIG. 15. Overview of interaction partners of human, murine, and zebrafish ZNG1 as identified by yeast-two-hybrid data.

FIG. 16. Structural statistics for NMR solution structure of the fusion of ZNG1₁₀₋₃₀ with METAP1-59.

FIG. 17. Proteomics hits from low zinc diet.

FIG. 18. Primers.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter relates to articles and methods for inhibiting methionine aminopeptidase (METAP) activity. In some embodiments, the method includes inhibiting METAP1 activity. In some embodiments, for example, the method includes targeting Zn regulated GTPase metalloprotein activator 1 (ZNG1) family proteins. ZNG1 proteins are metallochaperones that deliver zinc to, or metalate, METAP1, thereby supporting the function of METAP1. As such, inhibiting ZNG1 decreases or eliminates delivery of zinc to METAP1, which consequently decreases or inhibits METAP1 activity.

In some embodiments, the method includes targeted inhibition of ZNG1. For example, in some embodiments, the method includes inhibition of ZNG1 in specific cells, such as cancer cells. Any suitable method of targeting ZNG1 may be used, such as, but not limited to, using small molecules, dietary zinc interventions, or interference with the interaction of ZNG1 and METAP.

In some embodiments, the method includes inhibition of METAP activity by targeted inhibition of the interaction of ZNG1 and METAP to reduce or inhibit metalation of METAP. The targeted inhibition may occur at a specific interaction site between the two proteins.

Also provided herein are methods of treating a disease involving METAP1. In some embodiments, the method includes administering one or more inhibitors of ZNG1 to a subject in need thereof. In some embodiments, the one or more inhibitors are targeted. The disease includes any suitable disease involving METAP1, such as, but not limited to, cancer, obesity, diseases involving rapidly proliferating cells, metabolic syndromes, or any other suitable disease. In some embodiments, the method also includes administering one or more METAP2 inhibitors in conjunction with the one or more inhibitors of ZNG1. Without wishing to be bound by theory, it is believed that targeting ZNG1 as a modulator of cellular proliferation and lipid biosynthesis via METAP activity is a novel approach for anti-cancer and anti-obesity therapy. The methods of treating a disease involving METAP1 may in embodiments comprise any or all of the above-summarized methods.

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below. While the terms used herein are believed to be well understood by those of ordinary skill in the art, certain definitions are set forth to facilitate explanation of the presently-disclosed subject matter.

The terms “treatment” or “treating” refer to the medical management of a subject with the intent to cure, ameliorate, reduce, or prevent a disease or condition. As will be recognized by one of ordinary skill in the art, the term “cure” does not refer to the ability to completely eliminate a disease or condition. For example, in some embodiments, a cure can refer to a decrease at a level of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. Similarly, as will be recognized by one of ordinary skill in the art, the term “prevent” does not refer to an ability to completely remove any and all symptoms or evidence of a disease or condition.

Likewise, as will be recognized by one of ordinary skill in the art, the term “inhibiting” or “inhibition” does not refer to the ability to completely inactivate all target biological activity in all cases. Rather, the skilled artisan will understand that the term “inhibiting” refers to decreasing biological activity of a target, such as METAP, such as can occur, for example, when a nucleotide limits the expression of the target gene, when a ligand binding site of the target protein is blocked, or when a non-native complex with the target is formed. Such decrease in biological activity can be determined relative to a control, wherein an inhibitor is not administered and/or placed in contact with the target. For example, in some embodiments, a decrease in activity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. The term “inhibitor” refers to a compound of composition that reduces the expression of and/or decreases the biological activity of a target, such as METAP.

The terms “subject” or “subject in need thereof” refer to a target of administration, which optionally displays symptoms related to a particular disease, pathological condition, disorder, or the like. The subject of the herein disclosed methods can be a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “administering” refers to any method of providing a therapeutic composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable means such as intravenous administration, intra-arterial administration, intramuscular administration, peritoneal administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention or amelioration of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments+20%, in some embodiments+10%, in some embodiments+5%, in some embodiments+1%, in some embodiments+0.5%, in some embodiments+0.1%, in some embodiments+0.01%, and in some embodiments+0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### Examples

All combinations of method or process steps as used herein can be performed in any order, unless otherwise specified or clearly implied to the contrary by the context in which the referenced combination is made.

Experimental model and subject details

### Ethics Statement

Mouse and zebrafish studies were approved by the Institutional Animal Care and Use Committees of Vanderbilt University Medical Center (protocol numbers M1900043-00 and M1900076-00 respectively) in accordance with the Public Health Service Policy on the Human Care and Use of Laboratory Animals under the United States of America National Institutes of Health (NIH) Office of Laboratory Animal Welfare (OLAW).

### Zebrafish Husbandry

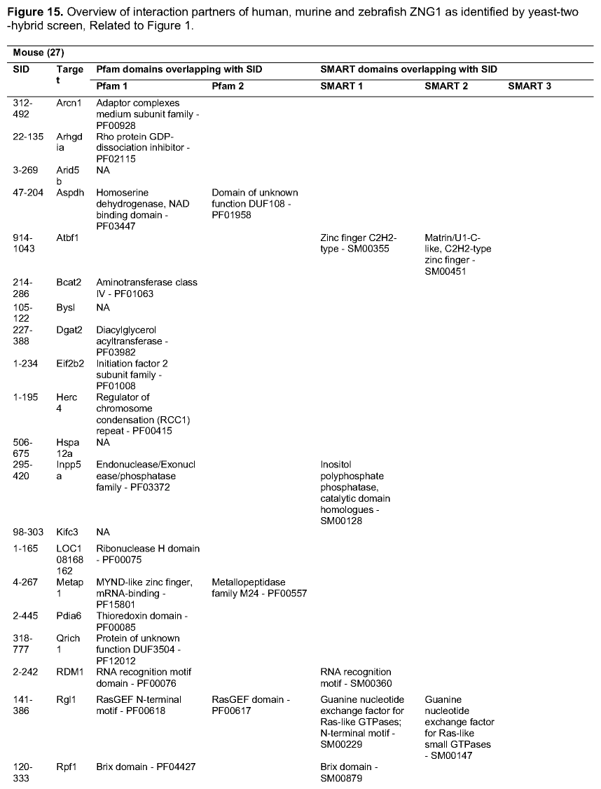

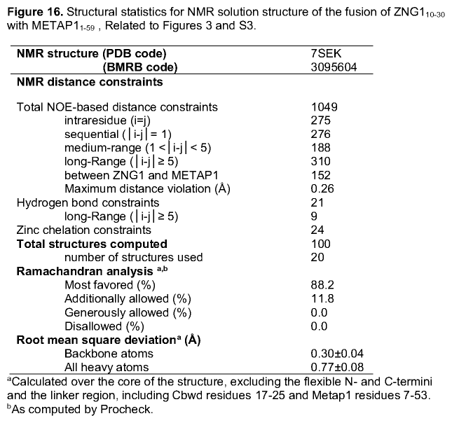

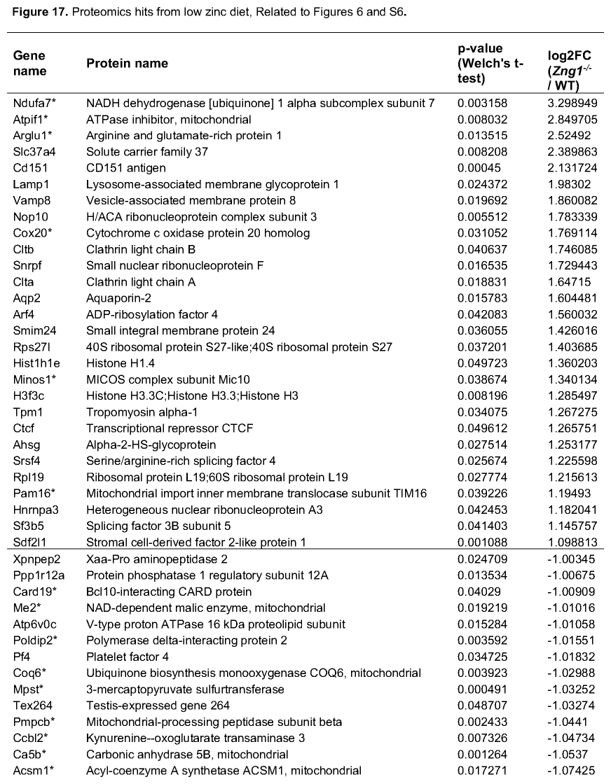

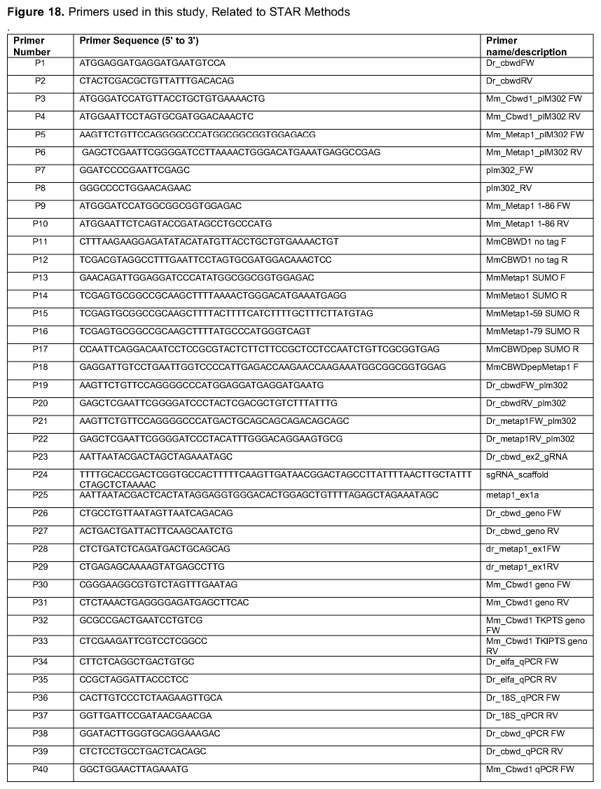

Yeast-two-hybrid data are provided within FIG. 15. Proteomics data are provided within FIGS. 17 and 18. Additionally, all mass spectrometry proteome data were submitted to ProteomeXchange via the PRIDE database under the submission reference number 562287. NMR structure information is provided within FIG. 16 and is deposited on PDB (7SEK).

All zebrafish lines were maintained on a mixed Tübingen (Tü)/AB background on a 14:10 h light: dark cycle in a recirculating aquaculture system. Embryos were collected from natural matings and maintained in embryo medium (0.03% Instant Ocean Sea Salt in RO water) at a density of ≤1 larva/mL at 28° C. 14:10 hour light: dark cycle. At 3 dpf, larvae were split randomly into treatment groups. All larvae used in experiments are of indeterminate sex.

### Mouse Husbandry and Diet

Experiments were performed using adult age-matched C57BL/6 (Jackson Laboratories) or C57BL/6 Zng1^{-/-} (breeding colony) mice. Animals were maintained at the Vanderbilt University Medical Center Animal Facilities and housed in groups of five. For each experiment, Zng1^{-/-} mice from multiple litters were included. For routine colony maintenance, mice were fed a standard chow diet (LabDiets; Rodent Chow Diet 5001). For manipulation of organismal Zn levels, mice were fed a defined Zn-free or control diet (Dyets Inc., AIN-93M Purified Rodent Diet with or without Zn supplementation at 29 parts per million). For experimental endpoints, animals were humanely euthanized. All animal experiments were approved and performed in compliance with the Institutional Animal Care and Use Committee (IACUC) of Vanderbilt University.

### Cell Culture Conditions

Mycoplasma-negative WT parental TKPTS and pooled Zng1 CRISPR/Cas9 mutant cells were ordered from Synthego (https://www.synthego.com/). Briefly, cells were propagated in DMEM/F-12 50/50 1X (Corning 10-092-CV) supplemented with 7% heat-inactivated fetal bovine serum (R and D Systems, S11150) with insulin (Sigma SLCF5002-0.3 mL of 10 mg/mL per 500 mL media) under 5% CO₂. Cells were passaged and trypsinized using TrypLE express (Thermo Fisher Scientific, 12605010).

### Method Details

### Yeast-Two-Hybrid Screening and Analysis

All yeast-two-hybrid screens were performed by Hybrigenics Services using full-length human ZNG1E, mouse ZNG1, and zebrafish Zng1. Murine Zng1 and human ZNG1E sequences were obtained from Genscript (Clone ID OMu12215 and OHu42907 in the pcDNA3.1-C-(k) DYK backbone, respectively). Full length zebrafish Zng1 was amplified from 6 days post fertilization (dpf) whole larval cDNA with P1 and P2 (Table S5) and cloned into pCRII TOPO (Invitrogen, K465001) and sequence verified by Sanger sequencing. The sequences described above were used as template for the generation of all subsequent ZNG1 constructs. Yeast-two-hybrid screens to identify interaction partners of ZNG1 proteins from different species were performed on a mixed (A549, H1703, H460) lung tumor cell library (human), an adult kidney library (mouse), or a whole embryo 20 h post fertilization library (zebrafish). Any interacting partners that were scored as experimental artifact were excluded from further analysis.

### Recombinant Protein Cloning, Expression, and Purification

### Cloning

Full-length murine Zng1 was amplified from pcDNA3.1-C-(k) DYK-Zng1 using primers P3 and P4 (Table S5). PCR products were inserted into the expression vector pLM302 (Center for Structural Biology, Vanderbilt University) containing a 3C protease cleavable N-terminal 6×His and Maltose Binding Protein (MBP) tag using BamHI and EcoRI restriction sites.

Full-length murine Metap1 was amplified from pcDNA3.1-C-(k) DYK-Metap1 (Gencript, Clone ID OMu05035) using primer pair P5/P6. Vector pLM302 was linearized by PCR (primers P7/P8, FIG. 18). Both amplicons were joined using the NEBuilder HiFi DNA Assembly Master Mix (New England Biolabs, E2621S).

A SUMO-tagged, truncated murine METAP1₁₋₈₆ was prepared by amplifying the N-terminal portion of the murine Metap1 construct using primers P9 and P10 (FIG. 18). The PCR product was cloned into the expression vector pBG102 carrying a 3C protease cleavable N-terminal 6×His and SUMO tag (Center for Structural Biology, Vanderbilt University) using the BamHI and EcoRI restriction sites.

For peptide, metal-binding, and METAP1 activation studies, murine ZNG1 was amplified using primers P11 and P12 before being cloned into the NdeI and BamHI sites in the pHis vector (Sheffield et al., 1999). Mouse Metap1, METAP1₁₋₅₉, and METAP1₁₋₇₉ were cloned into the NdeI and HindIII sites in the pSUMO vector (Peroutka lii et al., 2011) containing N-terminal 6×His and SUMO tags using primers P13-P16 (FIG. 18) via isothermal assembly (Gibson et al., 2009). The fusion of a 21-residue peptide from murine ZNG1 (ZNG1₁₀₋₃₀) onto the N-terminus of the truncated METAP1₁₋₅₉, used for the NMR structure, was generated by isothermal assembly (Gibson, 2011) of oligonucleotides P17 and P18 (FIG. 18) into the BamHI site of the METAP1₁₋₅₉ construct.

The full-length zebrafish zng1 coding sequence was cloned from 6 dpf whole larval cDNA using primers P1 and P2 into pCRII (Invitrogen) as described above (FIG. 18). Full-length zebrafish zng1 was subcloned from TOPO pCRII into pLM302 using primer pair P19/P20 (FIG. 16) and confirmed via Sanger sequencing. Full-length zebrafish metap1 was amplified from 6 dpf whole larval cDNA and cloned into pLM302 using primer pair P21/P22 (FIG. 18).

All constructs were confirmed by Sanger sequencing. Murine 6×His-SUMO-tagged METAP1₁₋₈₆ was transformed into E. coli C41 (DE3). All other constructs were transformed into E. coli BL21(DE3) for expression.

### Recombinant Protein Expression and Purification

All bacterial expression strains were grown aerobically in Lysogeny Broth (LB) while shaking. Depending on the specific expression vector, media were supplemented with antibiotic to ensure plasmid maintenance, including 50 μg/mL kanamycin, 100 μg/mL ampicillin, or 35 μg/mL chloramphenicol. Bacterial cultures were grown at 37° C. to OD₆₀₀=0.6. Protein expression was initiated with the addition of 0.2-1 mM isopropyl β-D-1-thiogalactopyranoside (IPTG), the temperature was lowered to 18° C., and the cultures allowed to grow for 14-16 h. Cells were harvested by centrifugation at 4,500×g for 15 min at 4° C. and stored at −20° C. prior to protein isolation.

For affinity chromatography, size exclusion chromatography, and dynamic light scattering experiments, the protein was isolated as follows. Bacterial pellets were resuspended in 10 mL lysis/wash buffer (20 mM HEPES (pH 7.5), 150 mM NaCl). Lysozyme (Thermo Fisher Scientific, 89833) was added at a final concentration of 1 mg/mL before incubating on ice for 10 min. Cells were lysed by sonication and cellular debris removed by centrifugation (4° C., 10,000×g, 30 min). The supernatant was loaded on a pre-washed column containing amylose resin (New England Biolabs, E8021). Columns were washed with 10 volumes of lysis/wash buffer before elution of bound protein with lysis/wash buffer containing 10 mM maltose. Protein purity and quantity was assessed by SDS-PAGE and BCA assay (Pierce, 23225 and 23209). To remove the 6His-MBP tag, protein was incubated with HRV 3C protease (Pierce, 88947) for 12-16h (4° C. with agitation). Cleaved tag was removed by loading the mixture on a pre-washed column containing HisPur Cobalt Resin (Thermo Fisher Scientific, 89964). The flow through was collected and protein purity and quantity assessed by SDS-PAGE and BCA assay (Pierce, 23225 and 23209). Purified protein was used immediately or flash frozen and stored at −80° C.

For peptide-binding, metal-binding, and METAP1 activation assays, ZNG1 and METAP1 were prepared as follows. Cells expressing untagged ZNG1 were resuspended in 25 mM Tris (pH 8.0), 500 mM NaCl, 2.5 mM EDTA, and 5 mM tris(2-carboxyethyl) phosphine (TCEP). Cells were lysed by sonication for 15 min, and the resulting lysate was centrifuged at 13,000×g for 20 min at 4° C. to remove cellular debris. The resulting supernatant was treated with 0.015% polyethylenimine on ice to precipitate nucleic acids, which were removed by centrifugation at 13,000×g for 20 min at 4° C. The supernatant was treated with 40% ammonium sulfate on ice for 30 min to precipitate ZNG1. The mixture was separated by centrifugation at 13,000×g for 20 min at 4° C., and the resulting pellet containing ZNG1 was resuspended in 25 mM Tris (pH 8.0), 150 mM NaCl, 2.5 mM EDTA, and 5 mM TCEP. This solution was dialyzed into 25 mM Tris (pH 8.0), 50 mM NaCl, 2.5 mM EDTA, and 5 mM TCEP overnight, injected onto a preequilibrated HiTrap Q FF (Cytiva Life Sciences, 17515601), and eluted with a gradient from 50 mM to 1 M NaCl. Fractions of >90% purity were combined for additional purification by size-exclusion chromatography (HiLoad 16/600 Superdex 200 pg, Cytiva Life Sciences 28989335) in 25 mM Tris (pH 8.0), 150 mM NaCl, 2.5 mM EDTA, and 5 mM TCEP. All fractions of >95% purity (by inspection of SDS PAGE gels) were collected and buffer-exchanged 10⁶-fold into chelex-treated (Biorad 141253) 25 mM 4-(2-hydroxyethyl)-1-piperazineethanesulfonic acid (HEPES (pH 7.4)), 150 mM NaCl, and 5 mM TCEP to create the final prep of ZNG1. ZNG1 prepared in this way was devoid of bound metal (<0.01 mol equiv of Zn (II) measured by ICP-MS).

For SUMO-tagged METAP1 constructs, cells were resuspended in 25 mM Tris (pH 8.0), 500 mM NaCl, and 5 mM TCEP. Cells were lysed by sonication for 15 min, and the resulting lysate was centrifuged at 13,000×g for 20 min at 4° C. to remove cellular debris. The resulting supernatant was injected onto a HisTrap FF (Cytiva Life Sciences, 17525501) column and eluted with a gradient from 0 to 500 mM imidazole. Fractions of >90% purity were combined and treated with SUMO protease for 2 h at room temperature to remove the N-terminal His-SUMO tag and dialyzed into 25 mM Tris (pH 8.0), 500 mM NaCl, and 2 mM TCEP. The cleaved protein product was injected onto a HisTrap FF column and the flow through was collected and concentrated for additional purification by size-exclusion chromatography (HiLoad 16/600 Superdex 200 pg for full-length METAP1 or HiLoad 16/600 Superdex 75 pg for truncated METAP1) in 25 mM Tris (pH 8.0), 150 mM NaCl, 2 mM TCEP. All fractions of >95% purity (by inspection of SDS PAGE gels) were collected and full-length protein was treated with chelator as indicated below. Purified protein was buffer-exchanged 10⁶-fold into chelexed 25 mM HEPES (pH 7.4), 150 mM NaCl, and 2 mM TCEP to create the final prep of METAP1, METAP1₁₋₅₉, or METAP1₁₋₇₉. Notably, we experienced significant decreases in METAP1 enzymatic activity from frozen protein stocks frozen over extended amounts of time (>1 month) and aliquots that had gone through multiple freeze thaw cycles. Therefore, final preparations of full-length METAP1 utilized in in vitro experiments in this study were purified, stripped of metal, and aliquoted for −80° C. storage all within one day. Moreover, frozen aliquots were thawed no more than twice to ensure maximal METAP1 enzymatic function. Proteins were analyzed by ICP-MS to identify metal stoichiometries (Figure S2F). To remove excess metal, METAP1 was treated with either 2 mM EDTA, 10 mM NTA, or 4 molar equivalents N,N,N′,N-terakis-(2-pyridylmethyl)ethylenediamine (TPEN) for 1 hour at 4° C. and then buffer-exchanged 10⁶-fold into chelexed 25 mM HEPES (pH 7.4), 150 mM NaCl, and 2 mM TCEP. To load Zn(II) into METAP1₁₋₅₉ and METAP1₁₋₇₉, 50 μM Zn(II) and 1 mM NTA were added to protein solutions for 1 hour at 4° C. and then buffer-exchanged 10⁶-fold into chelexed 25 mM HEPES (pH 7.4), 150 mM NaCl, and 2 mM TCEP. Molar extinction coefficients at 280 nm (35410 M⁻¹ cm⁻¹ for ZNG1, 55350 M⁻¹ cm⁻¹ for METAP1, 6990 M⁻¹ cm⁻¹ for METAP1-59, and 17990 M⁻¹ cm 1 for METAP1₋₇₉) were used to calculate protein concentration.

Uniformly ¹⁵N- and ¹⁵N¹³C-labeled METAP1₁₋₅₉ and the ZNG1₁₀₋₃₀-METAP1₁₋₅₉ fusion were prepared for NMR experiments using the SUMO tagged constructs as described above, except for the following details: The expression strains were grown in M9 minimal media with 1 g/L ¹⁵N ammonium chloride (Cambridge Isotope Laboratories) as the sole nitrogen source and either 4 g/L unlabeled glucose or 2 g/L ¹³C-glucose (Cambridge Isotope Laboratories) as the sole carbon source. 10% ¹³C-labeled protein was prepared using 1 g ¹⁵NH₄Cl, 0.4 g ¹³C₆-glucose, and 3.6 g unlabeled glucose. The lysis buffer was supplemented with EDTA-free protease inhibitor cocktail (Roche). Nickel-loaded HisTrap FF columns were used to isolate the protein from clarified lysate and after cleavage of the SUMO tag. To ensure that the metal binding sites were homogeneously occupied by Zn(II), the protein was exchanged 10³-fold into Zn-loading buffer (25 mM HEPES, 150 mM NaCl, pH 7.4, treated with chelex and supplemented with 1 mM nitrilotriacetic acid [NTA], 50 μM Zn(II), and 2 mM TCEP) using an Amicon ultra centrifugal filter unit (3 kDa cut-off), prior to 10³-fold exchange into NMR buffer (chelex-treated 10 mM sodium phosphate, 150 mM NaCl, pH 7.0, supplemented with 2 mM TCEP). These samples were confirmed by ICP-MS to contain 2.0 equivalents of Zn(II) and less than 0.01 equivalent of Ni(II).

### Affinity Chromatography to Determine ZNG1-METAP1 Interaction

To probe the interaction of the full-length proteins, His-tagged-MBP-ZNG1 (Mouse) was isolated as described above. Protein was loaded onto a previously equilibrated Ni-NTA column (Cytiva Lifesciences, 17-5248-05). The column was washed three times with five column volumes of lysis/wash buffer (10 mM Tris (pH 8) 150 mM NaCl). Following the washes, purified METAP1 with the tag cleaved was loaded onto the column and washed three additional times. After washing, the complex was eluted using wash buffer containing 500 mM imidazole. Fractions were visualized on a 4-20% Bis-Tris SDS-PAGE gel. Western Blot (methods described below) using a polyclonal METAP1 antibody (PA5-58202, 1:1,000) was performed to confirm identity of METAP1.

To test the interaction of zebrafish Zng1 and Metap1, His-tagged-MBP-Metap1 was isolated as described above, cleaved with HRV 3C protease (Pierce, 88947) passed through a HisPur Cobalt Resin (Thermo Scientific, 89964) column, and dialyzed in lysis/wash buffer overnight at 4° C. Subsequently, His-tagged-MBP-Zng1 was purified as described above with the following exception. Following flow through and washing of the amylose resin (New England Biolabs, E8021), purified Metap1 was passed over the column containing immobilized MBP-Zng1 or empty resin as control. Columns were subsequently washed with 100 mL of lysis/wash buffer prior to elution and fractions collected. Western blot detection of Zng1 (anti-CBWD1, HPA042813) of wash and elution fractions was performed using methods described below. Densitometry of Zng1 signal in each fraction was measured using Fiji software (Schindelin et al., 2012).

### Size Exclusion Chromatography to Determine ZNG1-METAP1 Interaction

The interaction of full-length murine ZNG1 with METAP1₁₋₈₆ was performed by size exclusion chromatography. Recombinant proteins were purified using methods described above and incubated for 1 h at 4° C. followed by passing over a Superdex 200 increase 10/300 GL column (Cytiva Lifesciences, 28990944). The buffer contained 50 mM Tris (pH 8) and 300 mM NaCl and the experiment was performed with a flow rate of 0.5 mL/min. The resulting fractions were separated and visualized by SDS-PAGE.

### Immunoblot Detection of ZNG1 and METAP1

For protein isolation, WT and Zng1 mutant TKPTS cells were washed once with warm PBS, trypsinized as described above, and transferred into fresh conical tubes. Samples were centrifuged at 200×g for 10 min. Cells were subsequently washed once with PBS and resuspended in 500 μl of cold RIPA buffer (Thermo Fisher Scientific, 89900) supplemented with Halt protease inhibitor (Thermo Fisher Scientific, 78442). Cells were subsequently vortexed for 10 sec and incubated on ice for 20 min prior to centrifugation for 15 min at 15,000×g. Supernatant was transferred into fresh Eppendorf tubes and protein concentration was measured by BCA assay (Thermo Fisher Scientific, 23225). Samples normalized for protein input were run on SDS PAGE gels in 1X Tris/Glycine/SDS Buffer (Bio-Rad, 1610732) and transferred using a semi-dry method using Bio-Rad trans blot turbo (25V, 1A, 18 min) onto a nitrocellulose membrane (Li-COR, 926-31092). Transfer efficiency was evaluated using staining with Ponceau S solution (Sigma P7170) following manufacturer's protocol. Membranes were washed three times in PBS supplemented with 0.2% tween (PBS-T), rocking for 5 min at room temperature. Blocking was performed using Odyssey (PBS) blocking buffer (Li-COR 927-40000) for 1 h at room temperature and subsequently washed three times with PBS-T. Primary antibodies were applied (anti-CBWD1, Sigma, HPA042813,1:500 dilution; anti-Tubulin, Cell signaling, 3873S, 1:1,000 dilution; anti-METAP1, Thermo Fisher Scientific PA5-58202, 1:1,000) in Odyssey blocking buffer and incubated overnight at 4° C. rocking. Membranes were washed three times with PBS-T for 5 min at room temperature. Secondary antibody solutions (Li-COR), species specific IR800CW or IR680LT, 1:2,000) were applied in Odyssey (PBS) blocking buffer, and membranes were incubated in the dark for 2 h rocking at room temperature. Following secondary antibody staining, membranes were washed three times with PBS-T and visualized using a ChemiDoc MP Imaging System with appropriate channels for detection.

### Murine METAP1 Methionine Aminopeptidase Assay

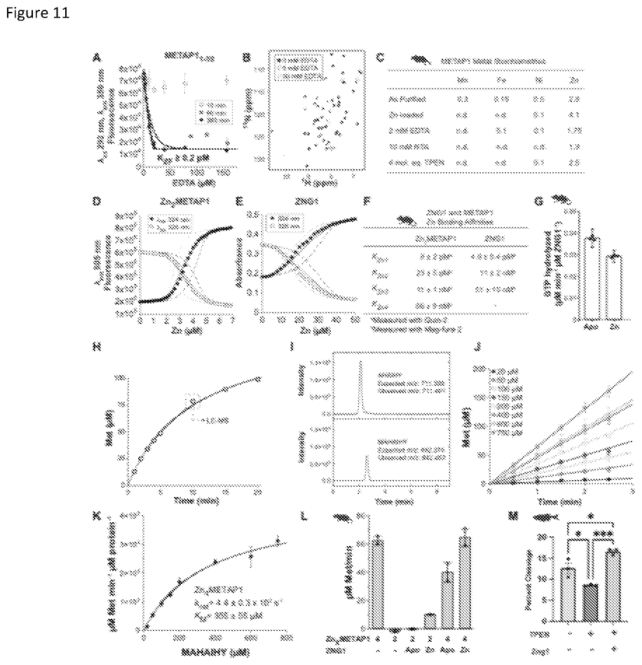

Methionine aminopeptidase activity was evaluated by quantifying the release of Met from the heptapeptide MAHAIHY (Genscript) as adapted from (Kabir-ud-Din, 2003). Reactions were conducted at 25° C. in 25 mM HEPES pH 7.4, 150 mM NaCl, 2 mM TCEP and consisted of 20 nM murine METAP1 with 0, 1, or 2 molar equivalents of Zn. Reactions were initiated with the addition of varying concentrations of MAHAIHY peptide. 50 μL aliquots were taken every 30 sec from 30-150 sec and the reaction was quenched with the addition of 10 μL glacial acetic acid (Supelco, Sigma-Aldrich). 60 μL of 4% w/v ninhydrin (Sigma-Aldrich) in 100% ACN was added to each quenched reaction and incubated at 37° C. for 1 h to detect free Met. The absorbance at 570 nm was measured and the cleaved Met concentration was determined from a standard curve of 10-500 μM Met. All data are reported as the average of triplicate measurements from each evaluated experimental condition. Midpoint and completed reactions were additionally analyzed by LC-ESI-MS to confirm the generation of the expected AHAIHY cleaved peptide product (FIG. 11 I).

### Murine ZNG1 Activation of METAP1 Assay

Activation assays were performed in 25 mM HEPES pH 7.4, 150 mM KCl, 2 mM MgCl2, 2 mM TCEP. 250 μM NTA was included where indicated. 10 μM Zn₂METAP1 was incubated with 25 μM apo or ZnZNG1 (ZNG1 loaded with 0.9 eq, 22.5 μM, Zn) at 37° C. for 1 h. Reactions were diluted 500-fold in 25 mM HEPES pH 7.4, 150 mM NaCl, 2 mM TCEP to a final concentration of 20 nM METAP1 and methionine aminopeptidase activity was assayed as described above by initiating the enzymatic reaction with addition of 750 μM MAHAIHY. When using GTP, GDP, or GDPNP, the activation reaction contained 500 μM of the indicated nucleotide. A control activation reaction of Zn-NTA contained 22.5 μM and 250 μM NTA in reaction buffer.

### Zebrafish Metap1 Methionine Aminopeptidase Activity

A single endpoint assay was used for these experiments. Reaction buffer (20 mM HEPES (pH 7.5), 150 mM NaCl) was chelex treated overnight at 4° C. 1 μM solutions of zebrafish Metap1 and Zng1 were made in reaction buffer. To strip Zn from the METAP1 active site, 3 μM of TPEN was incubated with 1 μM METAP1 at 4° C. for 1 h. To prepare holo-Zng1 (Zn bound), Zng1 was incubated with 10 μM of ZnCl₂ for 1 h at 4° C. As a control, Metap1 was incubated with reaction buffer alone. Following the 1 h incubation, Zng1 and Metap1 were washed four times with chelex-treated reaction buffer using protein concentration columns (Amicon Ultra, UFC500396). After washes, proteins were mixed and incubated at 37° C. for 30 min. Following incubation, 100 μM of the synthetic peptide MAHAIHY (Genscript) was added to reactions and incubated for 90 min at 37° C. Reactions were stopped by the addition of a final concentration of 2% trifluoroacetic acid and samples were centrifuged at max speed for 5 min. Clear supernatant was transferred into fresh glass vials for HPLC analysis.

Twenty μL of each sample and standard was analyzed on an Agilent 1260 Infinity II system. Analytes were separated by gradient HPLC on a Supelco Ascentis Express C18 column (50×2.1 mm, 5 μm) with a Phenomenex SecurityGuard C18 cartridge (3.2×8 mm) at a flow rate of 0.4 mL/min using 0.1% trifluoroacetic acid in water and 0.1% trifluoroacetic acid in acetonitrile as the A and B mobile phases, respectively. The gradient was held at 0% B for 2 min, then ramped to 100% B over the next 18 min. The column was washed at 100% B for 5 min, then equilibrated to 0% B for 5 min. MAHAIHY and AHAIHY were detected using absorbance at 280 nm, and retention times were confirmed using each standard. Methionine aminopeptidase relative activity was determined by calculating the percent of MAHAIHY converted to AHAIHY using the AUC values at 280 nm for each peptide.

### Dynamic Light Scattering

Purified and tag-free murine ZNG1 and METAP1 were incubated for 1 h at 4° C. before the experiments. The individual proteins and the mixture were filtered through 0.1 μm syringe filters to remove dust and trace residual lipid vesicles. Experiments were performed at 25° C. and data were analyzed using Dynamics 7.5 software (Wyatt Technologies). The DLS software calculates an apparent hydrodynamic radius (Rh) via the Stokes-Einstein equation (Stetefeld et al., 2016).

### Mag-Fura-2 Zn(II) Competition Assays

Experiments were performed using an HP8453 UV-vis spectrophotometer or an ISS PC1 spectrofluorometer at 25.0° C. Apo-mf2 (Invitrogen) was monitored by measuring the absorbance at 366 nm or by fluorescence emission at 505 nm upon excitation at 366 nm. Zn-mf2 was monitored by measuring the absorbance at 324 nm or the emission at 505 nm upon excitation at 324 nm. Several [protein]/[mf2] values were used for replicate experiments and analyzed globally to better calculate the stoichiometry. In a typical fluorescence experiment, 1 μM protein and 1 μM mf2 in 2.5 mL were incubated for 10 min in chelexed buffer [25 mM HEPES (pH 7.4), 150 mM NaCl, and 2.5 mM TCEP], and aliquots of Zn(II) were added. The equilibrium time was 2 min between subsequent additions of metal. The peak intensities at 324 and 366 nm from triplicate experiments were globally fit to 1:1, 2:1, 3:1, and 4:1 binding models, with the K_{Zn} value of mf2 fixed to 5×10⁸ M⁻¹ using Dynafit (Kuzmic, 1996). For ZNG1, the 3:1 binding model provided the best fit of the mf2 data alone, whereas the 4:1 binding model provided the best fit of the METAP1 mf2 data. The selected 4:1 binding model was subsequently used for a simultaneous global fit of triplicate mf2 data sets along with triplicate quin2 datasets for METAP1.

### Quin-2 Zn(II) Competition Assays

Experiments were performed using an ISS PC1 spectrofluorometer at 25.0° C. by monitoring the fluorescence emission of quin2 at 490 nm upon excitation at 353 nm, with quenching of the 490 nm emission maximum as aliquots of Zn(II) were added. Experiments were performed in triplicate at multiple protein: quin2 ratios. In a typical experiment, 1-2 μM protein and 1-2 μM quin-2 (Sigma 08520) were incubated for 10 min in chelexed buffer [25 mM HEPES (pH 7.4), 150 mM NaCl, and 2.5 mM TCEP] at a total volume of 3 mL, and 0.3 μM aliquots of Zn(II) were added. The equilibrium time was 12 min between subsequent additions of metal. The peak emission intensities at 490 nm from triplicate experiments were globally fit along with triplicate mf2 datasets using Dynafit (Kuzmic, 1996), with the K_{Zn} value of quin2 fixed to 2× 1011 M⁻¹

### Intrinsic Trp Fluorescence Quenching Assays

Peptide binding experiments were performed using an ISS PC1 spectrofluorometer at 25.0° C. Intrinsic Trp fluorescence was monitored using λₑₓ=292 nm nm and λₑₘ=350 nm, with quenching of the 350 nm emission maxima as aliquots of ZNG1 peptide were added. Different [METAP1_{1-59/1-79}] values were used for replicate experiments to better calculate the binding stoichiometry and analyzed globally. In a typical experiment, 5 μM METAP1_{1-59/1-79} in 2.5 mL were incubated for 10 min in chelexed buffer [25 mM HEPES (pH 7.4), 150 mM NaCl, and 2.5 mM TCEP], and aliquots of ZNG1 peptide were added. The equilibrium time was 2 min between subsequent additions of ZNG1 peptide. The peak emission intensities at 350 nm from triplicate experiments were globally fit to a 1:1 binding model using Dynafit (Kuzmic, 1996).

EDTA titrations of METAP1₁₋₅₆ were performed using in a Synergy Neo2 plate reader to monitor Trp fluorescence using Δₑₓ=292 nm and λₑₘ=350 nm for a series of EDTA (Alfa Aesar A107130B) concentrations ranging from 0.3 μM to 5 mM, for incubation times ranging from 10 min to 6 h at 25° C. Intensities from triplicate experiments were globally fit to a 2:1 Zn: METAP1₁₋₅₆ binding model using DynaFit to give an upper bound estimate of the affinity (Kuzmic, 1996) with the K_{Zn} value of EDTA fixed to 7.6×10¹² M⁻¹.

### ZNG1 Peptide Synthesis

The peptides were prepared as C-terminal amides and acetylated at the N-terminus by Fmoc solid-phase synthesis on Chem matrix Rink-Amide resin and purified by reverse-phase HPLC. Peptides were dried and resuspended in water. Calculated molar extinction coefficients at 214 nm were used to determine peptide concentration.

### NMR

### Assignments

NMR samples contained 0.5-2 mM uniformly ¹⁵N- and ¹⁵N¹³C-labeled protein, with 10% v/v D20 and 0.3 mM 2,2-dimethyl-2-silapentanesulfonic acid (DSS) as an internal reference. An equivalent 2 mM, ¹⁵N¹³C-labeled 100% D20 sample was prepared by lyophilization of a 100% H₂O sample, followed by resuspension in D20 (Cambridge Isotope Laboratories). NMR spectra were recorded at 25° C. on a Bruker Avance Neo 600 MHz spectrometer equipped with a cryogenic probe or on a Varian VNMRS 800 MHz spectrometer with a room temperature 5 mm PFG HCN probe in the METACyt Biomolecular NMR Laboratory at Indiana University, Bloomington. Data were collected using Topspin 4.0.9 (Bruker) or VNMRJ 4.2 (Varian), processed using NMRPipe (Delaglio et al., 1995), and analyzed using CARA (http://cara.nmr.ch/doku.php) and NMRFAM-Sparky (Lee et al., 2015), all on NMRbox (Maciejewski et al., 2017).

Backbone chemical shifts were assigned for each state using the following standard triple-resonance experiments: HNcaCB, CBCAcoNH, and HNCO (Sattler, 1999). Chemical shift assignments were not obtained for the C′ of K59 or C22 in the free or bound states because an HNCO does not include resonances for C′ in terminal residues or in residues preceding proline. An HNcaCO of the fusion construct provided these chemical shifts as well as those of C18 and V22 in the ZNG1 peptide, which also precede proline residues. With one proline in the zf-C₆H2 domain and two proline residues in the peptide, 100% of all possible amide ¹H, ¹⁵N atoms are assigned in all three states. 97% of all possible C′ atoms are assigned in the free and bound forms, and 100% in the fusion. 100% of all possible Ca and Cb atoms are assigned in the free domain and fusion, but 98% of all Ca and Cb atoms in the bound form, without assignments for C22 due to chemical exchange broadening. The backbone assignments of METAP1₋₅₉ in the absence of peptide are consistent with and expand upon those previously published for human METAP1₁₋₈₃ (Rachineni et al., 2015).

Aliphatic side-chain assignments were obtained using HBHAcoNH, HcccoNH-TOCSY, hCccoNH-TOCSY, HcCH-TOCSY, hCCH-TOCSY experiments (Sattler, 1999), while hbCBcgcdHD, hbCBcgcdceHE, and H^{ar} (CC-TOCSY-CGCBCACO) NH for were used for aromatic side-chains (Lohr et al., 2007; Yamazaki T, 1993). The 3D experiments were collected using non-uniform sampling with Poisson gap scheduling (Hyberts et al., 2010), and reconstructed with istHMS (Hyberts et al., 2012). Sidechain assignments of the peptide-zf-C₆H2 fusion construct are essentially complete, with the assignment of the single methionine methyl group confirmed by a 3D ¹³C-NOESY-HSQC. Stereospecific assignments of methyl groups for all 3 valine residues and all 5 leucine residues in the fusion construct were obtained using a combination of high-resolution ¹H¹³C-HSQC and constant-time ¹H¹³C-HSQC of a 10% ¹³C-labeled sample (Hilty et al., 2003). The chemical shifts and protonation states of the Nd and Ne atoms of the two histidine sidechains were assigned using a ¹H¹⁵N-HSQC experiment optimized for ²J_{NH} transfer (Pelton et al., 1993), demonstrating that the Nd atoms are protonated and the Ne atoms coordinate Zn^{II}. A small number of non-amide exchangeable protons were observed in 3D ¹⁵N-NOESY-HSQC spectra of the fusion construct, and were attributed to the OSH hydroxyl groups of Ser58, Ser65, and Thr68. Chemical shifts were analyzed for indications of secondary structure using TALOSN (Shen and Bax, 2015). All backbone and side chain chemical shifts have been deposited in the BioMagResBank under accession number 51117 (free), 51118 (bound), and 30956 (fusion) (Ulrich et al., 2008).

### Dynamics

NMR relaxation measurements including ¹⁵N spin relaxation rates, R₁ and R₂, and ¹H-¹⁵N heteronuclear NOE (hNOE) values were recorded in an interleaved manner using standard experiments. The relaxation delays used were 0.02, 0.06, 0.10, 0.20, 0.40, 0.60, 0.80, and 1.2 s for R₁ and 0.017, 0.034, 0.051, 0.068, 0.085, 0.102, 0.119, 0.136, 0.153, 0.170, 0.204, and 0.238 s for R₂. Residue-specific R₁ and R₂ values were obtained from fits of peak intensities vs. relaxation time to a single exponential decay function, while hNOE ratios were ascertained directly from intensities in experiments recorded with (2 s relaxation delay followed by 3 s saturation) and without saturation (relaxation delay of 5 s). Errors in hNOE values were calculated by propagating the error from the signal to noise. Backbone relaxation rates have been deposited in the BioMagResBank under accession numbers 51117 (free) and 51119 (fusion).

### Structure Determination

Initial structure calculations were performed with CYANA using automatically assigned NOESY peak lists with no further restraints, revealing the expected cross-brace zinc chelation topology (Guntert, 2004). Zn coordination geometry was defined and maintained for subsequent calculations using distance restraints (Zn—Sg=2.3 Å and Zn—Ne2=1.95 Å). Hydrogen bond restraints were added where supported by NOEs, chemical shift-based secondary structure predictions, and slow rates of hydrogen-deuterium exchange. The structure has been deposited into the Protein Data Bank with accession ID 7SEK.

### GTPase Activity Measurements (Malachite Green Assay)

Reactions were run in 25 mM HEPES pH 7.4, 150 mM KCl, 2 mM MgCl2, 2 mM TCEP. 20 μL reactions of 5 μM ZNG1 in the presence or absence of 5 μM Zn and 5 μM Zn₂METAP1 were initiated with the addition of 500 μM GTP (Sigma 10106399001) and incubated at 37° C. for 90 min. GTPase reactions were quenched and inorganic phosphate was detected with the addition of 120 μL of 1 mM malachite green oxalate (Acros Organics), 10 mM ammonium molybdate (Sigma-Aldrich) in 1 M HCl. This reaction was incubated in the dark at ambient temperature for 10 min and then quenched with the addition of 60 μL 35% citric acid. Mixtures were incubated for 10 min and inorganic phosphate concentration was calculated based on the absorbance at 680 nm relative to a standard curve (5-500 UM phosphate, Fluka Analytical).

### GTPase Activity Measurements (Coupled Assay)

GTPase activity was measured following a previously described protocol (Ingerman and Nunnari, 2005). Reactions were run in 25 mM HEPES pH 7.4, 150 mM KCl, 2 mM MgCl2, 2 mM TCEP in a total volume of 100 μL and included 4 μM ZNG1, 500 μM GTP (Sigma 10106399001), 260 μM NADH (Sigma N8129), 2 mM phosphoenolpyruvate (Sigma 860077), and 1 μL pre-mixed Pyruvate Kinase/Lactate Dehydrogenase (Sigma P0294) at 37° C. Reactions were monitored in real time by a decrease in absorbance at 340 nm in a Synergy Neo2 plate reader.

### Zebrafish Mutagenesis

Targeted deletion of the zng1 and metap1 genes were performed using CRISPR/Cas9 gene editing targeting the second exon of zng1 and first exon of metap1 as described elsewhere (Essner, 2016). Briefly, the guide RNA sequences 5′ GACCCACAGCTCAGATCC 3′, targeting zebrafish zng1 and 5′ AGGTGGGACACTGGAGCT 3′ targeting metap were designed using CRISPRscan (Moreno-Mateos et al., 2015) and were synthesized using the oligo-based method (Yin et al., 2015) (P23-P25 Table S5). Cas9 mRNA was generated from XbaI (New England Biolabs) digested pT3TS-nls-zCas9-nls plasmid (Addgene, 46757), and in vitro transcribed using mMESSAGE mMACHINE T3 kit (Thermo Fisher Scientific, AM1348) (Jao et al., 2013). A cocktail consisting of 150 ng/μL of nls-zCas9-nls and 120 ng/μL of gRNA, 0.05% phenol red, 120 mM KCl, and 20 mM HEPES (pH 7.0) was prepared, and approximately 1-2 nL was injected directly into the cell of one cell stage Tübingen zebrafish embryos. Mutagenesis was initially screened using Melt Doctor High Resolution Melting Assay (Thermo Fisher Scientific, 4409535), and identified two independent zng1 alleles which were confirmed as deletions by Sanger sequencing of TOPO-cloned PCR products. Subsequent screening of the 411 (allele designation vu2) was performed by PCR (primers P26/P27, FIG. 18) and products were resolved on 2% agarose Tris-Borate-EDTA (TBE) gels. Screening of the 45 allele (allele designation vu1) was performed by PCR amplification using primers P26 and P27 (FIG. 18), followed by purification and DpnII digest (New England Biolabs). All zng1 mutant alleles result in the loss of a single DpnII restriction site present in the WT sequence. Sanger sequencing identified a 17 base pair deletion in the metap1 first exon (allele designation vu3) which was genotyped by PCR using P28/P29 (Table S5) and resolved on 2% agarose Tris-Borate-EDTA (TBE) gels.

### Zebrafish Pharmacological Manipulations

For all experiments larval zebrafish were bred naturally and collected into embryo media and were maintained at 28° C. on a 14/10 h light/dark cycle for duration of experiments. For survival experiments, zebrafish larvae were split into even densities into 6 or 12 multi-well plates in 5 mL or 1 mL embryo media respectively at 3 dpf. Stock solutions of METAP2 inhibitor TNP-470 (Sigma, T1455) and METAP1/2 inhibitor Bengamide B (Santa cruz, sc-397521A) were prepared in ACS-grade DMSO (Fisher Scientific, D136) and TPEN (Sigma, P4413) was prepared in ethanol and stored at −20° C. TNP-470, Bengamide B, and/or TPEN were applied into embryo media as well as a DMSO and/or ethanol vehicle controls at 3 dpf. Zebrafish survival was monitored daily until 6 dpf and all moribund or dead animals were removed. For qPCR experiments, larvae were split at 3 dpf into groups of 20-30 larvae in 20 mL of embryo media supplemented either 20 μM of TPEN or ethanol vehicle control. For imaging experiments, 6 dpf zebrafish larvae treated with 500 nM to 1 μM of Bengamide B or vehicle control were imaged by a Leica Thunder M205FCA stereomicroscope with a Leica DFC9000 GT camera.

### Mouse Mutagenesis

Generation of Zng1^{-/-} mice. Zng1^{-/-}-mice were produced using CRISPR-Cas9 genome editing in a C57B1/6J strain (The Jackson Laboratory) through the Vanderbilt Genome Editing Resource (Vanderbilt University). Ribonucleoprotein complexes containing crRNA+tracrRNA (ctRNA) and SpCas9 protein (MilliporeSigma) targeting DNA sequences within Zng1 exon 1 and a single stranded DNA donor containing an EcoRI restriction site were delivered by pronuclear injection into 1-cell C57BL/6J embryos. crRNA sequences were crRNA: 5′TGGAGTCACTGGTGTCCTGC crRNA: 5′CCGGGTACTTAGGTAATTAA* and 180 nucleotide ssODN donor sequence: ctagaagagaggccccagcccctacctcgaagattcgtcctcggccaggaggcccgggtgcctgaggagacacggcagcccccctta GAATTCggacaccagtgactccagaactaaagagctgccgccagtcgcgtttccgcgacaggattcagtcggcgcggggctgatg atgtcacc. F0 founder animals were screened for precise Zng1 deletions by standard PCR and restriction fragment length polymorphism assays. Deletions were confirmed by Sanger sequencing and verified by a second round of Sanger sequencing in the heterozygous N1 generation. N1 generation Zng1^{-/-} heterozygous mice containing the desired mutation were backcrossed at least three generations into the C57B1/6J strain to allow for segregation of potential off-target editing events prior to familial breeding to produce homozygous offspring. Genotyping of mice from the Zng1 mutant breeding colony was performed by Transnetyx using ear punches of individual animals and primer pair P30/P31 (Table S5). Zng1^{-/-} animals will be cryopreserved and are available through the Vanderbilt Cryopreserved Mouse Repository.

### Cell Line Mutagenesis

Targeted deletion of the first exon of murine Zng1 was performed using the guide RNA ‘ATTCCAGTCACAATTGTCAC’. Clonal selection of mutant cells was performed by dilution plating. Individual clonal populations of cells were subsequently screened using primers P32 and P33 (FIG. 18) followed by Sanger sequencing of TOPO-cloned PCR products. Two independent mutant clones were identified and harbored a homozygous Δ2/Δ2 base pair deletion (clone 1) and a 47/42 base pair deletion (clone 2).

### Mass Spectrometry Proteomics of Murine Tissue and Cell Lines

Kidneys were isolated from 11-13-week old C57BL/6 or C57BL/6 Zng1^{-/-} mice that were fed a low Zn diet for 5 weeks. Animals were humanely euthanized, kidneys isolated, and flash frozen in liquid nitrogen. For protein isolation, frozen organs were gently thawed on ice and transferred through a 70 μM sterile cell strainers (Thermo Fisher Scientific, 22-363-548) into 5 mL of ice-cold PBS supplemented with Halt protease inhibitor (Thermo Fisher Scientific, 78430). Filters were flushed an additional two times with PBS with protease inhibitor. Cells were pelleted (4° C., 400× g, 10 min) and pellets resuspended in 500 μL of RIPA buffer with Halt protease inhibitor (Thermo Fisher Scientific, 78430). Following rigorous vortexing, incubation on ice (10 min), and a second round of vortexing, cellular debris were pelleted by centrifugation (4° C., 10,000× g, 10 min) and supernatant transferred into fresh tubes for further analysis.

Protein concentrations were determined using the 660 Protein Assay (Pierce, 22660). Equal amounts of protein (200 μg) were processed for LC-MS/MS using s-traps (Protifi) (HaileMariam et al., 2018; Zougman et al., 2014). Briefly, proteins were reduced with dithiothreitol (DTT), alkylated with iodoacetamide (IAA), acidified using phosphoric acid, and combined with s-trap loading buffer (90% MeOH, 100 mM TEAB). Proteins were loaded onto s-traps, washed, and finally digested with Trypsin/Lys-C (1:100, w: w; enzyme: protein) overnight at 37° C. Peptides were eluted and dried with a vacuum concentrator. Peptides were resuspended in H₂O/1% acetonitrile/0.1% formic acid for LC-MS/MS analysis.

Peptides were separated using a 75 μm×50 cm C18 reversed-phase-HPLC column (Thermo Fisher Scientific, 164570) on an Ultimate 3000 UHPLC (Thermo Fisher Scientific) with a 120 min gradient (2-32% ACN with 0.1% formic acid) and analyzed on a hybrid quadrupole-Orbitrap instrument (Q Exactive Plus, Thermo Fisher Scientific). Full MS survey scans were acquired at 70,000 resolution. The top 10 most abundant ions were selected for MS/MS analysis.

Raw data files were processed in MaxQuant (v 1.6.14.0, www.maxquant.org) and searched against the current UniProt Mus musculus protein sequences database. Search parameters included constant modification of cysteine by carbamidomethylation and the variable modifications, methionine oxidation and protein N-term acetylation. Proteins were identified using the filtering criteria of 1% protein and peptide false discovery rate. Protein intensity values were normalized using the MaxQuant LFQ function (Cox et al., 2014).

Label free quantitation analysis was performed using Perseus (v 1.6.14.0), software developed for the analysis of-omics data (Tyanova et al., 2016). LFQ Intensity values were Log2-transformed, and then filtered to include proteins containing at least 60% valid values (reported LFQ intensities) in at least one experimental group. Finally, the missing values in the filtered dataset were replaced using the imputation function in Perseus with default parameters (Tyanova et al., 2016). Statistical analyses were carried out using the filtered and imputed protein groups file. Statistically significant changes in protein abundance are determined using Welch's t-test P-values and z-scores.

### Mass Spectrometry Proteomic Quantification of Zng1 Peptides

Murine brain tissue was homogenized in NP40 buffer (50 mM trizma base, 150 mM sodium chloride, 1 mM EDTA, and 1% nonidet 40) using cycles of sonication. Protein concentration was quantified using a Bicinchoninic Acid assay (Sigma Aldrich, QPBCA). For each sample, 10 μg of protein was suspended in 8 M urea containing 50 mM ammonium bicarbonate to a final volume of 90 μL. Proteins were reduced using a final concentration of 20 mM dithiothreitol (DTT, Thermo Scientific A39255) at 37° C. for 45 min, followed by alkylation using 20 mM iodoacetamide (IAA, Thermo Scientific A39271) at room temperature for 15 min. Samples were diluted to reduce the concentration of urea to <1 M using 50 mM ammonium bicarbonate and proteins were digested using 1 μg of LysN (Thermo Scientific 90300) at 50° C. for 2 h. Oasis mixed-mode cation exchange (MCX) solid phase extraction cartridges (Waters Corporation, Milford, MA 186000252) were used to remove lipids and other contaminants from the digested peptides. Peptide samples were dried using a centrifugal evaporator.

Dried digested samples were reconstituted in HPLC-grade water containing 0.1% formic acid by vortexing and centrifuged briefly to remove any particulates. A total of 250 ng of each sample was desalted and concentrated using C18 EvoTips (EvoSep EV2001). Liquid chromatography mass spectrometry was performed using an EvoSepOne (EvoSep) coupled to an Orbitrap Fusion (ThermoFisher Scientific) equipped with a Nanospray Flex Ion source. Chromatography was performed using a 15 cm long column containing ReproSil-Pur C18 1.9 μm beads by Dr Maisch C18 and a fused silica emitter tip (20 μm inner diameter) (EvoSep EV1112, EV1111). Peptides were eluted from the column using an EvoSep 20 samples/day Whisper method. Mass spectrometry was performed on an Orbitrap Fusion (ThermoFisher Scientific) equipped with a Nanospray Flex Ion source. MS1 was performed using Orbitrap detection at a resolution of 60,000 with a standard AGC target. Data independent acquisition was performed using quadropole isolation with nonoverlapping windows of 16 m/z from precursor masses 400-1008 with HCD fragmentation (33% collision energy) and Orbitrap detection with a resolution of 30,000. The normalized peak areas for Zng1 peptides were calculated using Skyline (MacLean et al., 2010).

### Gene Expression Analysis

For RNA isolation from dissected mouse tissue, mice were humanely euthanized, tissues harvested and immediately flash frozen in liquid nitrogen and stored at −80C. Mouse tissues were transferred into Lysing matrix B tubes (MP, 6911100) containing 700 μl of RLT buffer (Qiagen, 74104) with 1% (v/v) B-mercaptoethanol. Samples were homogenized by bead beating 6 times for 40 s at 6 m/s. Tubes were kept on ice for 1 min in between steps. Samples were centrifuged and homogenate was added to a fresh tube containing 600 μl of phenol: chloroform: iAA (Fisher Scientific, BP17521-100) then vortexed for 30 s. Samples were spun at max speed for 15 minutes and the aqueous phase was transferred to a tube containing 700 μl of 100% ethanol. Subsequent isolation steps were performed using the Qiagen RNeasy Kit following manufacturer's protocol (Qiagen). gDNA was depleted from final RNA samples by treatment with TURBO DNA-free Kit (Invitrogen). For RNA isolation from zebrafish, pools of 20-30 whole larvae were collected into 1 mL of TRIzol (Thermo Fisher Scientific, 15596026) and stored at −80° C. For TKPTS cell lines, cells were washed with PBS and collected with 500 μL TRIzol from multi-well plates and stored at −80° C. Zebrafish larvae and cell lines were homogenized by passing samples 10-15 times through a 27-gauge needle. RNA was isolated following the manufacturer's protocol with the following modification: a second wash with 70% ethanol (prepared in DEPC-treated H2O) was performed. RNA was treated with DNaseI following manufacturer's instructions (New England Biolabs, M0303L). cDNA from RNA isolated by any of the methods described above was synthesized using the iScript kit (Bio-Rad, 1708891). Quantitative PCR was performed in duplicate-quadruplicate 25 μL reactions using 2× iQ SYBR Green SuperMix (BioRad, 1708882) run on an BioRad CFX96 Real-Time System instrument using gene-specific primers (zebrafish: P34-P39, mice: P40-P43, cell lines: 44-69, Table S5). Data were analyzed with the ΔΔCt method (Livak and Schmittgen, 2001) or presented as relative expression values.

### Transfection of Zng1 Mutant Cells

To generate linearized DNA for transfection, pcDNA3.1-C-(k) DYK-Zng1ₘᵤᵣᵢₙₑ was digested with ScaI. Cells were grown to 80-90% confluency in 6-well TC treated plates and transfected with lipofectamine reagent using manufacturer's protocol (Lipofectamine 3000, Invitrogen L3000-008). Briefly, 2.5 μg of linear DNA was mixed with P3000 reagent and opti-MEM media (Gibco, 11058-021) to prepare the DNA solution. DNA solution and lipofectamine reagent solutions were mixed at a 1:1 ratio and incubated for 15 min at room temperature. DNA-lipid complexes were added to cells dropwise and cells were incubated overnight at 37° C. at 5% CO₂. G418 (RPI G64500-10) was added at a concentration of 300 μg/ml in complete DMEM/F12 50/50 complete cell culture media described above. G418 resistant cells were diluted and clonally expanded to identify clonal populations of transfected cells. Expression of full-length ZNG1 was validated using Western blot against ZNG1.

### Immunofluorescence

WT and Zng1 mutant TKPTS cells were seeded onto sterile coverslips in 6-well tissue culture treated multi-well plates. At 60-80% confluency, cells were washed three times with warm phenol red free DMEM (Thermo Fisher Scientific, 21041025) prior to fixation with 4% paraformaldehyde solution (PFA, Alfa aesar, J61899) for 15 min at 37° C. then washed three times for 5 min with PBS. Cells were subsequently permeabilized by incubation in 0.2% Triton X-100 in PBS for 10 min at room temperature. Following permeabilization, cells were washed three times for 5 min with PBS at room temperature. Cells were blocked using Odyssey (PBS) blocking buffer (LI-COR) for 1 h at room temperature or overnight at 4° C. Primary antibodies were diluted in Odyssey blocking solution and incubated overnight at 4° C. rocking (anti-OxPhos Complex V inhibitor Clone 5E2, Invitrogen A21355, 1:200). Cells were washed three times with PBS for 5 min. Secondary antibodies (Thermo Fisher Scientific) and Hoechst (Invitrogen, H3570) were added to Odyssey blocking buffer (Hoechst, 1:1,000, Alexa-conjugated secondaries 1:2000) and cells were incubated for 2 h rocking at room temperature. Following secondary staining, cells were washed three times with PBS and mounted onto slides with 20 μL of ProLong Gold antifade reagent (Invitrogen, 2273617). Slides cured in the dark overnight at room temperature prior to imaging. Slides were imaged on the Zeiss LSM 880 using the 63X oil objective (1.40 Plan-APOCHROMAT OIL, WD=0.19 mm).

### Transmission Electron Microscopy

WT and Zng1 mutant TKPTS cells treated with 3 μM TPA (Sigma, 723134) were fixed in 2.5% glutaraldehyde in 0.1 M cacodylate for 1 hour at room temperature followed by 24 hours at 4C. The cells were sequentially postfixed in 1% tannic acid, followed by 1% OsO4, then enblock stained with 1% uranyl acetate. Samples were dehydrated in a graded ethanol series and gradually infiltrated with a Quetol 651 formulation Spurrs resin using propylene oxide as the transition solvent. The resin was polymerized at 600C for 48 hours. Thin sections were cut using a Leica UC7 ultramicrotome with a nominal thickness of 70 nm and collected onto 300 mesh copper grids. All samples were post-stained with 2% uranyl acetate and lead citrate.

TEM imaging was performed on a Tecnai T12 operating at 100 keV using an AMT nanosprint CMOS camera. Single images were acquired using the AMT Capture Engine software. Images for quantification were acquired using serialEM to acquire montages of entire gridsquares, using identical lens currents between samples. These montages were reconstructed using the IMOD/etomo software suite. Mitochondria size, shape, and electron density quantification was performed using the FIJI ROI manager to manually segmentation all mitochondrial cross-sections within randomly selected cells until at least 100 mitochondria were measured for each treatment.

### Flow Cytometry Measurement of Cell Proliferation and Mitochondrial Superoxide

TKPTS cells were seeding at 2,000 to 4,000 cells per well of 24- or 12-well tissue culture treated multi-well plates and incubated overnight at 37° C. Cells were staining with CellTRACE violet (Invitrogen, C34557) as described by manufacturer's protocol. Briefly, CellTRACE solution was prepared in PBS at a 1:1,000 dilution, added to the cells, and cells were incubated for 20 min at 37° C. Cells were then washed two times in cell culture media supplemented with 7% FBS, and replaced with fresh cell culture media supplemented with desired chemical treatments (3 μM TPA, Sigma, 723134; 5 μg/ml TNP470, Sigma, T1455; or 50 nM Bengamide B, Santa cruz, sc-397521A). Stock solutions of drugs were prepared in ACS-grade DMSO (Fisher Scientific, D136). For growth in Zn deplete conditions, TPA was chosen because it exhibits lower cytotoxicity towards cell lines compared to other chelators (e.g. TPEN) (Huang et al., 2013; Lo et al., 2020). After three days of growth, cells were stained with MitoTracker far-red (Invitrogen, M22426; 1:2500 dilution for 20 min at 37° C.) and MitoSOX red (Molecular probes, M36008: 1,1000 for 20 min at 37° C.) following manufacture's protocols. Cells were washed with PBS, trypsinized, and transferred to Eppendorf tubes prior to fixation in 4% PFA (Alfa aesar, J61899). Cells were fixed for 20 min at room temperature, centrifuged at 200×g for 10 min and washed with PBS prior to resuspension in 300 μl FACS buffer (2% FBS, 0.02% sodium azide in PBS). All flow cytometry data were collected using a BD LSRII flow cytometer with FACSDIVA software and analyzed using FlowJo (FlowJo LLC). Samples were gated forward scatter height (FSC-H) by forward scatter area (FSC-A) and side scatter height (SSC-H) by side scatter area (SSC-A) to remove doublet populations. The singlet population was gated SSC-A by FSC-A to isolate live cells. The resulting cell population was then assessed for assay-specific fluorescent markers and the median fluorescence intensity (MFI) quantified.

### Seahorse XF96 Cell Mitochondrial Stress Test

8,000 WT or Zng1 mutant vehicle treated TKPTS cells or TKPTS cells serially passaged in 3 μM TPA (Sigma, 723134) were plated in individual wells of seahorse plates in complete DMEM F12 media described above. Cells were resuspended in mitochondria assay media (Seahorse Agilent pH 7.4 media+1 mM pyruvate+10 mM glucose+2 mM glutamine). No TKPTS cells were added to the four wells in the corners of the plate as a background control. For the mitochondrial stress test, the cartridge was sequentially loaded with oligomycin (15 μM), FCCP (15 μM), and rotenone/antimycin A (5 μM) (diluted in mitochondria assay media) for injection into the wells. To normalize across wells by cell density, the Seahorse plate was imaged using a BioTek Cytation 5. Oxygen consumption and extracellular acidification rates were collected using a Seahorse XFe96 Analyzer and analyzed using Wave Desktop software (Agilent Technologies).

Measuring cellular ATP production

Cellular ATP levels were measured from WT and Zng1 mutant TKPTS cell lysates following manufacturer's protocols (Sigma, MAK113). Briefly, 20,000 cells/well WT and Zng1 mutant cells were seeded in clear-bottom black 96-well plates (Costar, 3603) and incubated for 36 h at 37° C. under 5% CO₂. ATP levels were measured using Cell Titer Glo 2.0 (Promega, G9241). To normalize to cell number, cells were incubated with Hoechst (1:1,000 in PBS) for 15 min at 37° C. in cell culture incubator then washed with PBS prior to the addition of 100 μL of fresh PBS. Hoechst signal was measured in the BioTek Cytation 5 (ex/em 350/461).

Subsequently, 100 μL of Cell Titer Glo reagent was applied directly to the wells and incubated in the dark for 15 min at room temperature. Luminescence was measured in the BioTeck Cytation 5 and expressed as relative light units (RLU).

### Tetramethylrhodamine Methyl Ester (TMRM) Assay

WT and Zng1 mutant TKPTS cells untreated or treated with 3 μM TPA (Sigma, 723 134) were seeded at a density of 4,000 cells/well in clear-bottom black 96-well plates (Costar, 3603) and incubated overnight at 37° C. under 5% CO₂. Adherent cells were stained with MitoTracker deep red (Invitrogen, M22426) as described above. TMRM (Invitrogen, T668) staining solution was prepared following manufacturer's instructions. Briefly 100 mM TMRM was prepared in phenol red-free cell growth medium and was added to adherent cells at a quenching concentration of 200 nM as described previously (Monteith et al., 2013). Cells were incubated for 1-2 h at 37° C. at 5% CO₂. TMRM and MitoTracker fluorescence was measured using a BioTek Cytation 5 (TMRM-ex/em 548 nm/574 nm and MitoTracker-ex/em 644 nm/665 nm). For each genotype and treatment condition, 4-8 independent wells were analyzed and TMRM signal was normalized to MitoTracker signal.

### Determination of Metal Levels by Inductively Coupled Plasma Mass Spectrometry (ICP-MS)

Mouse tissue homogenate were acid digested in 400 μL of Optima grade nitric acid (Thermo Fisher Scientific, A467-500) and 100 μL of Ultratrace hydrogen peroxide (Sigma, 95321) at 65° C. for 48 h. After digestion, the acid content was diluted to below 10% with 3.5 mL of UltraPure water (Invitrogen, 10977-023). Elemental quantification on acid-digested samples was performed using an Agilent 7700 inductively coupled plasma mass spectrometer (Agilent) attached to a Teledyne CETAC Technologies ASX-560 autosampler (Teledyne CETAC Technologies). The following settings were fixed for the analysis Cell Entrance=−40 V, Cell Exit=−60 V, Plate Bias=−60 V, OctP Bias=−18 V, and collision cell Helium Flow=4.5 mL/min. Optimal voltages for Extract 2, Omega Bias, Omega Lens, OctP RF, and Deflect were determined empirically before each sample set was analyzed. Element calibration curves were generated using ARISTAR ICP Standard Mix (VWR, BDH82026-108). Samples were introduced by peristaltic pump with 0.5 mm internal diameter tubing through a MicroMist borosilicate glass nebulizer (Agilent). Samples were initially up taken at 0.5 rps for 30 s followed by 30 s at 0.1 rps to stabilize the signal. Samples were analyzed in Spectrum mode at 0.1 rps collecting three points across each peak and performing three replicates of 100 sweeps for each element analyzed. Sampling probe and tubing were rinsed for 20 s at 0.5 rps with 2% nitric acid between every sample. Data were acquired and analyzed using the Agilent Mass Hunter Workstation Software version A.01.02.

### Computational Tools

Sequence analysis, alignments and cladograms were created using CLC Genomics Workbench (version 20.0.1.). Visualization of pairwise alignments was generated using Sequence Demarcation Tool (SDTv1.2) (Muhire et al., 2014). Pathway analysis of proteins with significantly altered abundance (as determined by Welch's t-test) was performed using the Ingenuity Pathway Analysis tool (IPA, Qiagen). Structural multiple sequence alignments were generated using ESPript (Robert and Gouet, 2014). An iterative HMMsearch was used to identify variations in the ZNG1 N-terminal motif (Eddy, 2009). HMMsearch was also used to identify matches to the refined motif in yeast, human, mouse, and zebrafish genomes. Sequence logo motifs were generated using WebLogo 3.7.4 (Crooks et al., 2004). AlphaFold2 modeling of the yeast Map1p-Zng1p interaction was performed using the ColabFold AlphaFold2_advanced jupyter notebook with max_recycles set to 48 (Mirdita et al., 2021).

### Statistical Methods

All experiments were repeated at least two times and statistical analyses were performed with GraphPad Prism v.9 or OriginPro. Data are presented as mean±SEM unless otherwise noted in figure legend. For comparisons between 2 groups a two tailed student's t-test or Mann-Whitney test was applied. For comparisons between 3 or more groups, a one-way ANOVA with Tukey's multiple comparisons test was used. For experiments with 2 independent variables, a two-way ANOVA was performed with Tukey's or Sidak's multiple comparisons test. Significance was set as P<0.05, and denoted as: * P<0.05, ** P<0.01, *** P<0.001, ** P<0.0001. Sample sizes are indicated in the figure legends.

### Results

ZNG1s are Ancient COG0523 Proteins with Conserved Metalloprotein Interactions.

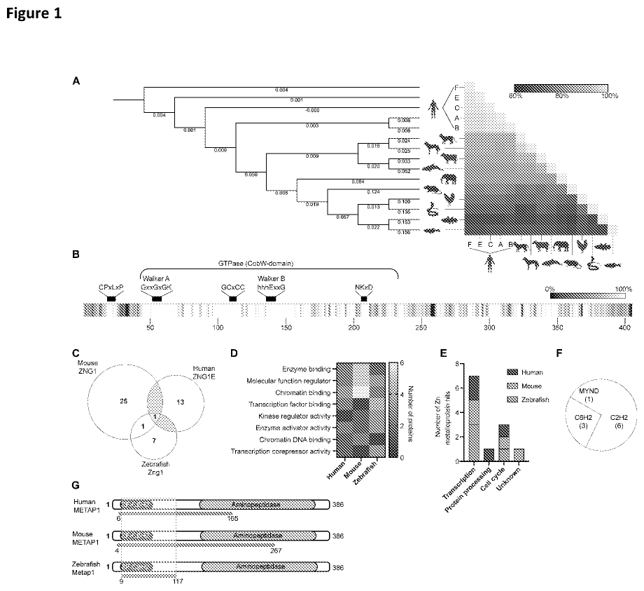

To interrogate the role of vertebrate ZNG1s in Zn homeostasis, the evolutionary conservation of ZNG1 proteins was evaluated by aligning ZNG1 amino acid sequences from vertebrates spanning an evolutionary distance of over 400 million years (Howe et al., 2021; Kumar et al., 2017). While many vertebrates, including mice and zebrafish, encode for a single ZNG1 protein, a chromosomal duplication event within the primate lineage led to the expansion of the COG0523 gene family to five intact ZNG1 paralogs (ZNG1A-F with ZNG1DP being a pseudogene) (Wong et al., 2004). There is over 60% sequence conservation across all analyzed ZNG1 proteins (FIG. 1A and S1A). Among the analyzed ZNG1 members, the known catalytic motifs and metal-binding residues of the GTPase domain are uniformly present and conserved (FIG. 1B) (Edmonds et al., 2021; Haas et al., 2009). Of note, several regions outside of the GTPase domain are also conserved, including the N-terminus (FIG. 1B).

Despite broad evolutionary conservation, the in vivo functions of COG0523 proteins, including ZNG1, have yet to be determined in part due to a lack of knowledge of interaction partners. To identify potential ZNG1 client proteins, three independent yeast-two-hybrid screens were performed with full-length ZNG1s as bait. To assess species-specificity as well as evolutionarily conservation of interactions, the screens were performed using zebrafish Zng1, mouse ZNG1, and human ZNG1E proteins. ZNG1E was chosen as the representative human protein due to its consistently high expression across different tissue types and high similarity to the consensus sequence of the human ZNG1 proteins (Figure S1B, C) (Uhlen et al., 2015). Of note, species-specific nomenclature referring to genes and proteins from the representative backgrounds will be maintained throughout the manuscript (e.g. human: ZNG1E, ZNG1E; mouse: Zng1, ZNG1; zebrafish: zng1, Zng1). For each screen between 9 and 27 putative ZNG1 interacting partners were identified (FIG. 1C, Table S1). Importantly, while each dataset revealed species-specific interaction partners, the Zn-finger protein methionine aminopeptidase 1 (METAP1) was identified in each of the three screens. These findings suggest that ZNG1 interacts with both common and distinct proteins across vertebrate species.

In addition to identifying interaction partners, the yeast-two-hybrid approach also discerned predicted Selected Interacting Domains (SID) of target proteins. SIDs were analyzed to identify the presence of specific protein domains (defined by PFAM or SMART) that are partially or completely contained within each SID (Table S1). To test if ZNG1 supports distinct cellular processes, an enrichment analysis of identified PFAM domains was performed using the dcGO platform (Fang and Gough, 2013). A high prevalence of domains with functions in protein and chromatin binding as well as factors with regulatory roles were identified (FIG. 1D). Since ZNG1 expression is associated with conditions of Zn limitation (Ogo et al., 2015), we hypothesized that ZNG1 aids in the mobilization of cellular Zn during limitation and that proteins with known Zn-binding domains would be interaction partners enriched in these data sets. Indeed, 12 Zn metalloproteins were identified across all three screens, including proteins involved in transcription, protein processing, and the cell cycle (FIG. 1E). For most of these proteins, the predicted site of interaction with ZNG1 harbors a Zn finger (zf) motif, including C₂H2 (Cys₂His₂), C₆H2 (Cys₂His₂, also referred to as MYND-like), or MYND (myeloid, Nervy, and DEAF-1) (FIG. 1F), consistent with known roles of zf domains in facilitating protein-protein interactions (Brayer et al., 2008; Fedotova et al., 2017; Gross and McGinnis, 1996). Because METAP1 was the single conserved ZNG1 interaction identified in all yeast-two-hybrid screens, the METAP1-ZNG1 interaction was selected for additional investigation. To further define the region of METAP1 that interacts with ZNG1, the results from the three screens were compared to predict a minimal METAP1-interacting region (FIG. 1G). This analysis suggested that an N-terminal segment of 108 residues within METAP1 containing the zf-C6H2 domain is the site of interaction with ZNG1.

The METAP1 zf-C6H2 Interacts with a Conserved Motif in ZNG1.

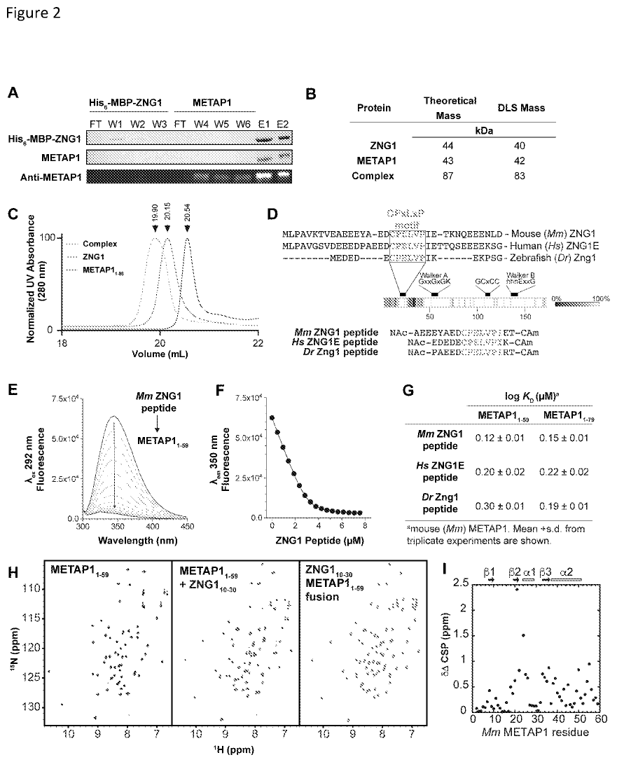

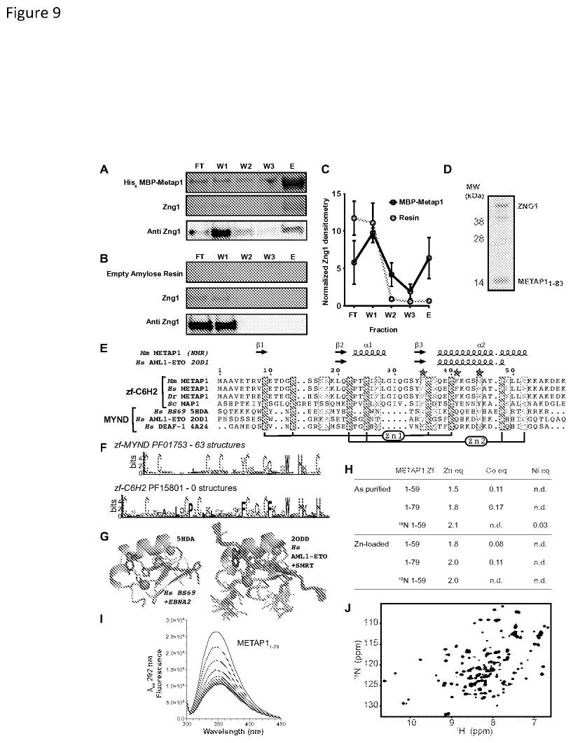

The interaction of full-length recombinant ZNG1 and METAP1 from mouse and zebrafish was confirmed by affinity chromatography (FIGS. 2A and 9A-C) and by dynamic light scattering (DLS) (FIG. 2B). Masses obtained from DLS for murine ZNG1 and METAP1 individually, as well as for the equimolar complex, were consistent with theoretical masses (FIG. 2B). Size exclusion chromatography was performed using murine ZNG1 and N-terminally truncated METAP1₁₋₈₆ containing the complete zf-C6H2 domain (FIG. 9D). The observed shift in elution volume of the 1:1 mixture, compared to individual proteins, indicates that the N-terminus of METAP1 is sufficient to mediate the interaction of ZNG1 with METAP1 (FIGS. 2C and 9D).

Determination of a minimal binding site on ZNG1 for the interaction with METAP1 was informed by the observation that zf-C6H2 domains resemble zf-MYND domains, with four pairs of conserved cysteine and histidine residues as well as conserved aromatic residues (Figure S2E-G). No structure has been determined for a zf-C6H2 domain, but structures of zf-MYND domains in AML1/ETO, DEAF-1, and BS69 reveal a ββα architecture, two Zn ions coordinated in an interdigitated pattern, and a binding interface for peptides containing a P×L×P motif (Figure S2E,G) (Harter et al., 2016; Kateb et al., 2013; Liu et al., 2007). The METAP1 zf-C6H2 domain also binds two Zn ions, even when truncated to METAP1₁₋₅₉ or METAP1₁₋₇₉, as measured by inductively coupled plasma-mass spectrometry (ICP-MS) (Figure S2H). Moreover, a conserved CPEL VPI sequence at the N-terminus of ZNG1 homologs matches the P×L×P motif that zf-MYND binds (FIGS. 1B and 2D).

METAP1 binding of ZNG1 peptides was confirmed by monitoring quenching of W45 fluorescence in murine METAP1₁₋₅₉ and METAP1₁₋₇₉, upon addition of chemically synthesized N-terminal ZNG1 peptides from human, mouse, and zebrafish (FIGS. 2D,E and 9 I). These data reveal a 1:1 binding stoichiometry and a K_{D}≈0.1 μM (FIG. 2F-G), which is ≥100-fold tighter than the zf-MYND domains with their cognate peptides (Kateb et al., 2013; Liu et al., 2007). Mouse, human, and zebrafish ZNG1 peptides bound with similar affinities to both METAP1₁₋₅₉ and METAP1₋₇₉ (FIG. 2G), indicating that the N-terminal 59 residues are sufficient for binding. Combined, these findings identify the specific regions of interaction between ZNG1 and METAP1.

NMR spectroscopy was used to confirm peptide binding and to explore the interaction in more detail. ¹H-¹⁵N HSQC spectra of ¹⁵N-enriched METAP1₁₋₅₉ show excellent signal dispersion and uniform intensities indicating a stable, well-folded domain (FIG. 2H, left). Addition of a half-molar equivalent of ZNG1 peptide produces a second set of peaks of equal intensity (FIG. 9 J), indicating a complex in the slow chemical exchange regime on the NMR time scale; addition of a full molar equivalent of ZNG1 peptide reveals a saturated complex. (FIG. 2H, middle panel). These results confirm tighter binding than previously observed for MYND domains (Kateb et al., 2013; Liu et al., 2007), and motivate a more detailed analysis of the peptide binding interface.

Solution Structure of the METAP1 zf-C6H2: ZNG1 Peptide Complex.

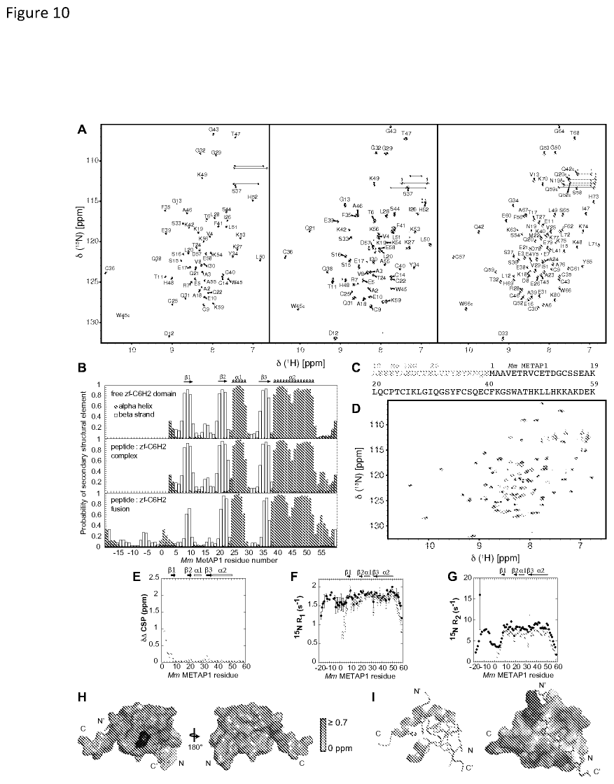

An NMR chemical shift perturbation assay was used to identify the METAP1 residues most directly involved in peptide binding. Complete backbone chemical shift assignments for both the peptide-free and peptide-bound states of METAP1₁₋₅₉ (Figure S3A) reveals that residues 21-24 are particularly affected by peptide binding, suggesting direct contact with the peptide (FIG. 21), while residues 1-18 and 27-32 are relatively unaffected and likely distant from the binding site. Further, chemical shift-based secondary structure predictions reveal that the METAP1 domain has a ββαβα architecture in the free and bound forms (FIG. 10B, top, middle), similar to but distinct from the ββα architecture of zf-MYND domains (FIG. 9E) (Shen and Bax, 2015). The secondary structure is minimally perturbed by peptide binding, as in zf-MYND domains (Liu et al., 2007).

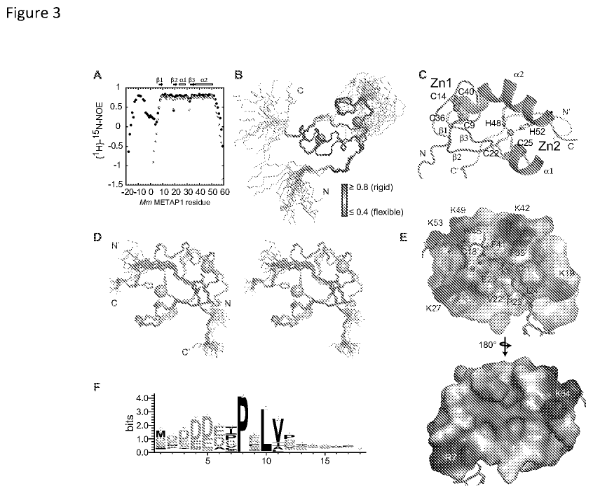

Further NMR studies of the bound form of METAP1₁₋₅₉ were not possible due to peak splitting for many residues, a limitation circumvented by studying a fusion construct inspired by the structure determination of the AML1/ETO zf-MYND bound to the SMRT peptide (Liu et al., 2007). The ZNG1 peptide was expressed at the N-terminus of METAP1₁₋₅₉ (FIG. 10C). The ¹⁵N-labeled fusion reproduces the spectra of the bound form, without the multiplets observed previously (FIGS. 2H and 10D). Minimal perturbations to chemical shifts, localized to the N-terminus of METAP1 validate the utility of the fusion construct (FIG. 10A right; 10B bottom; 10E). Moreover, backbone dynamics show that the linker region is flexible and that the dynamics of the domain are consistent in the free and fusion forms (FIGS. 3A,B and 10F,G). The central region of the peptide encompassing the CPELVPI sequence has elevated heteronuclear NOE values indicative of tight binding. Additionally, the fusion construct enables direct observation of the ZNG1 peptide resonances, facilitating structure determination. An initial, low-resolution 3D solution NMR structure of the ZNG1₁₀₋₃₀ METAP1₁₋₅₉ fusion relying exclusively on automated NOE assignments with no additional constraints clearly shows the same cross-brace Zn-binding topology as in a zf-MYND domain. Zn1 is coordinated by C9, C14, C36, and C40 and Zn₂ by C22, C25, H48 and H52 (FIG. 3C).

The final solution structure shows the novel zf-MYND-like fold, with an additional α-helix and β-strand (FIG. 3D, FIG. 16). This strand pairs with the C-terminal strand to form a 1,3,2 antiparallel B-sheet, in which strand 2 hydrogen bonds with the peptide, consistent with the pattern of ¹H, ¹⁵N chemical shift perturbations (FIG. 10H). The negatively charged peptide binds a hydrophobic groove in a positively charged face of the domain, surrounded by conserved lysine residues, K19, K27, K42, K49, and K53 (FIGS. 3E and 9E-F). In the hydrophobic groove, C18′ of ZNG1 contacts the W45 side chain in METAP1, while P19′ contacts F41, the L21′ sidechain inserts into the deep pocket lined by F35 and F41, the V22′ sidechain makes hydrophobic contacts with the conserved P23, and the 124′ sidechain packs against Y34. Sidechains of Q21 and Q38 in METAP1 form hydrogen bonds with the ZNG1 peptide backbone. Collectively, these data reveal the molecular features of the ZNG1-METAP1 interaction, showing that the high affinity comes from a combination of electrostatic and hydrophobic interactions, as well as hydrogen bonds, consistent with the conservation of these residues in METAP1 and in ZNG1.

### ZNG1 Supports METAP1 Function in a Zn-Dependent Manner.

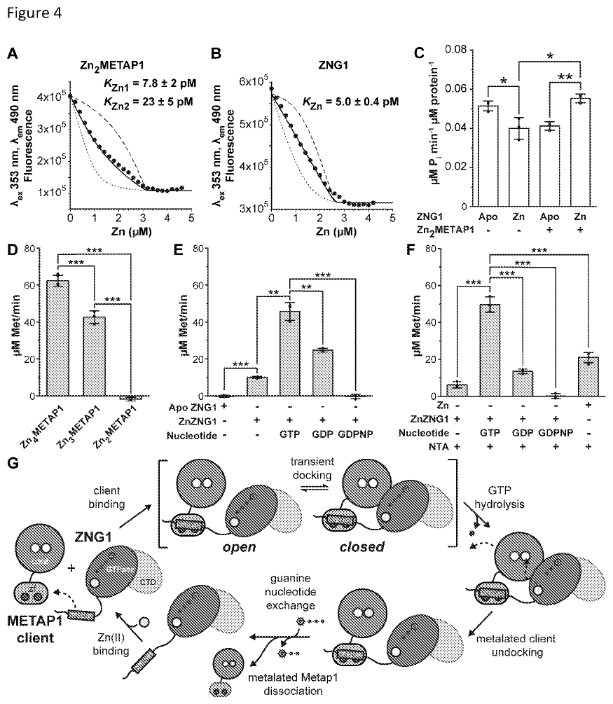

Based on our previous work with bacterial COG0523 proteins, we hypothesize that ZNG1 uses GTP hydrolysis to transfer Zn from the high affinity C×CC binding site into the METAP1 active site, thereby maintaining METAP1 activity under conditions of low cellular Zn. In addition to the two Zn ions coordinated by the zf-C6H2 domain, METAP1 binds two metal ions in the active site (Addlagatta et al., 2005). Recombinant murine METAP1 can therefore be loaded with Zn to four molar equivalents (termed Zn₄METAP1). Removal of Zn by extended exposure to EDTA disrupts the zf fold (FIG. 11A,B); however, gentler treatment with chelators including nitrilotriacetic acid (NTA; K_{Zn}≈10 nM) selectively removes metal from the active site while leaving the zf domain intact, generating Zn₂METAP1 (FIG. 11C). Mag-fura-2 (mf2) and quin-2 Zn competition experiments with Zn₂METAP1 show two high affinity (K_{Zn1}≈8 pM, K_{Zn2}≈20 pM) and two low affinity Zn binding sites (FIGS. 4A and 11D). Murine ZNG1 binds three molar equivalents of Zn(FIG. 11E), with one high affinity site (K_{Zn1}≈5 pM) likely coordinated by the C×CC motif in the core GTPase domain (FIG. 4B) (Jordan et al., 2019) and two low affinity sites. Like other COG0523 proteins of the same phylogenetic grouping (Edmonds et al., 2021), GTP hydrolysis is slow and slightly inhibited when Zn is bound solely at the high-affinity site (ZnZNG1) (FIG. 4C,11F) (Sydor et al., 2013). However, ZnZNG1 GTPase activity is stimulated by Zn₂METAP1, while ApoZNG1 is not (FIG. 4C).

To assess the effect of ZNG1 on METAP1 activity in vitro, we first determined initial rates of aminopeptidase activity for the various Zn-bound forms of METAP1 by quantifying released methionine (Met) from the heptapeptide MAHAIHY, a known METAP1 target (Xiao et al., 2010) (FIG. 11H-K). Active site-bound Zn is required for activity as Zn₂METAP1 exhibits no turnover, while Zn₃METAP1 partially recovers the activity of fully active Zn₄METAP1 (FIG. 4D). Incubation of ApoZNG1 and Zn₂METAP1 shows no activation of Zn₂METAP1, while addition of ZnZNG1 recovers small amounts of Zn₄METAP1 activity (FIG. 4E). ZnZNG1 shows no activation of fully metalated Zn₄METAP1 (FIG. 11L), consistent with a regulatory impact only on undermetalated METAP1. We next assessed the nucleotide dependence of ZnZNG1 activation of Zn₂METAP1 by incubation with GTP, GDP, or nonhydrolyzable GTP analog, GDPNP. In the presence of GTP, ZnZNG1 nearly fully activates METAP1, and GTP hydrolysis is required for this activation as ZnZNG1 in the presence of GDPNP exhibited no activation (FIG. 4E). Although GDP shows some ZnZNG1-dependent activation of METAP1 above that of ZnZNG1 alone, it is far less than that of GTP (FIG. 4E). This could result from a conformational change in ZNG1 after GTP hydrolysis that may facilitate release of Zn into the METAP1 active site (Jordan et al., 2019). We performed the same activation experiments in the presence of NTA to ensure Zn-dependent activation of Zn₂METAP1 is due to Zn transfer from ZNG1 directly to METAP1, rather than from Zn in solution. The GTP-bound form of ZnZNG1 again exhibits significant activation of Zn₂METAP1 above that of NTA-bound Zn, while GDPNP has no effect (FIG. 4F). Similarly, ZnZNG1 in the presence and absence of GDP shows only minor activation of Zn₂METAP1 activity (FIG. 4F). Zebrafish Zng1 also stimulates apo-Metap1 activity (Figure S4M). Together, these data establish that ZNG1 activates Zn₂METAP1 in a Zn- and GTP hydrolysis-dependent manner (FIG. 4G).

### ZNG1 Promotes In Vivo Metap Function in Zebrafish.

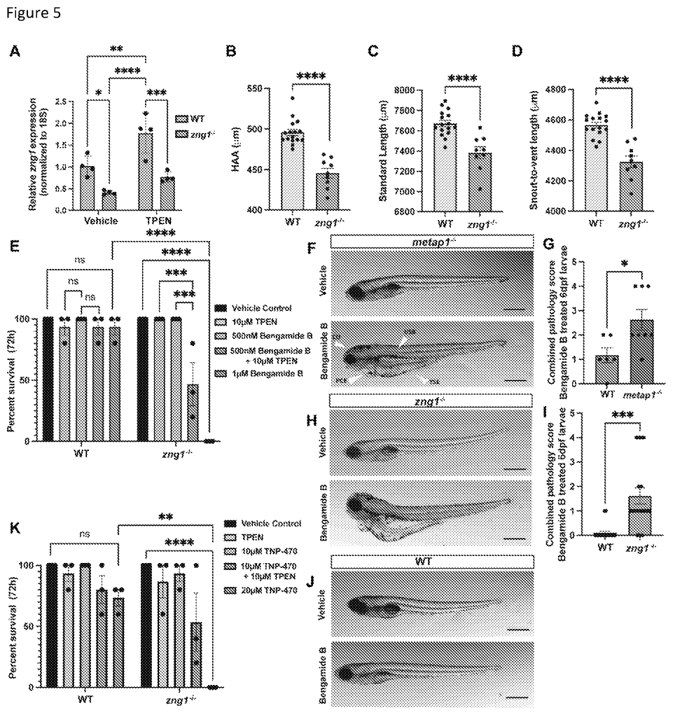

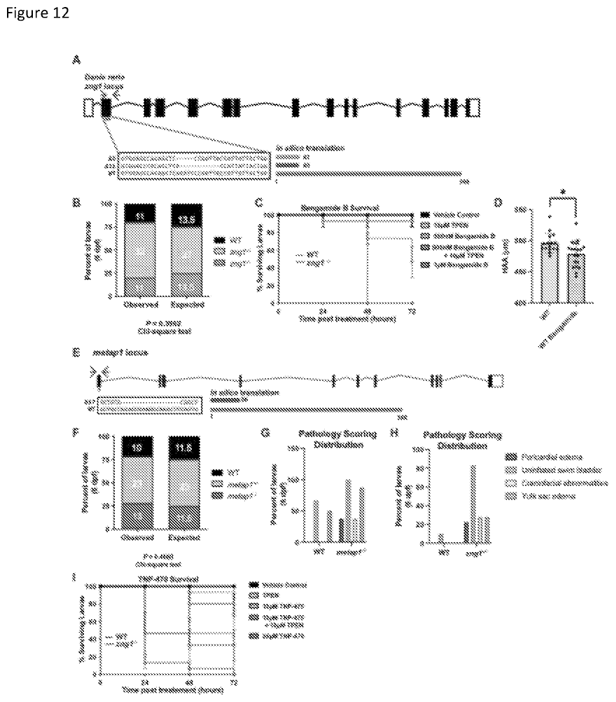

To study the in vivo role of Zng1, we turned to a zebrafish model. Zebrafish possess a single copy of zng1, which has high sequence similarity to mammalian ZNG1 homologs (FIG. 1A). Based on the reported Zn-dependent repression of ZNG1 (Coneyworth et al., 2012; Ogo et al., 2015), we first sought to assess zng1 expression in a zebrafish. To modulate organismal Zn levels, chemical treatment of zebrafish larvae was performed via immersion in system water containing the Zn chelator N,N,N′,N′-Tetrakis(2-pyridylmethyl)ethylenediamine (TPEN). Treatment of zebrafish larvae with TPEN resulted in a significant induction of zng1 transcript in WT larvae (FIG. 5A). To investigate Zng1-Metap1 interaction in vivo, zng1 mutant zebrafish (hereafter referred to as zng1^{-/-}) were generated using CRISPR/Cas9 gene editing (FIGS. 5A, 12A). zng1 mutant fish survived to adulthood under Zn-replete conditions yet morphometric analysis revealed zng1 mutant larvae exhibit moderate developmental delay with reduced height at anterior anal fin (HAA), standard length, and snout to vent length at 6 days post fertilization (dpf) (FIGS. 5B-D, 12B) (Parichy et al., 2009).

We hypothesized a role of Zng1 in maintaining Metap1 function and Zn homeostasis in zebrafish. Zebrafish encode for a single copy of Metap1 with high sequence similarity to mammalian METAP1, and two paralogs of Metap2 (Metap2a and Metap2b). Previous studies indicate functional redundancy between METAP1 and METAP2 suggesting that zebrafish Metap2 paralogs could compensate for Metap1 activity in zng1^{-/-} animals (Frottin et al., 2006; Li and Chang, 1995; Xiao et al., 2010). To investigate the effects of impaired Metap1 function on organismal health, Metap1 activity in vivo was modulated using pharmacologic inhibition of Metap enzymes in larval zebrafish. WT and zng1^{-/-} larvae were exposed to the established METAP1/2 inhibitor Bengamide B in Zn-deplete or replete conditions and survival was monitored over three days of treatment (Emilio Quinoa, 1986; White et al., 2017). zng1 mutant larvae displayed increased sensitivity to Bengamide B with 100% lethality when treated with 1 μM of inhibitor as compared to WT controls which remained viable (FIGS. 5E, 12C). We hypothesized that inhibition of Metap activity in zng1 mutant animals would be exacerbated in conditions of low Zn, consistent with in vitro data suggesting that Zng1 supports metallation of Metap1 under Zn limiting conditions. Indeed, co-treatment of zng1 mutant larvae with a sublethal dose of Bengamide B (500 nM) and TPEN resulted in decreased viability as compared to treatment with 500 nM Bengamide B alone (FIGS. 5E, 12C). Notably, WT animals treated with Bengamide B also displayed significantly reduced HAA (Figure S5D) mirroring the defects observed in zng1^{-/-} larvae, indicating a functional link between Zng1 and Metap1 in vivo. Combined, these data suggest Metap activity is impaired in zng1 mutant larvae, especially in Zn-deplete conditions.

To further evaluate Zng1 modulation of in vivo Metap1 activity, metap1 mutant zebrafish were generated by CRISPR/Cas9 (hereafter referred to as metap1^{-/-}-) (Figure S5E,F). As expected, pharmacologic inhibition of total Metap activity in metap1 “larvae resulted in significantly higher morphologic abnormalities as compared to WT controls at 6 dpf (FIG. 5F,G). These abnormalities included pericardial edema (PCE), yolk sack edema (YSE), uninflated swim bladder (USB), and craniofacial defects (CD) (FIGS. 5F, 12G) (Raghunath and Perumal, 2018). Importantly, zng1 mutant larvae exhibited similar defects when exposed to the maximum tolerable dose of Bengamide B (FIGS. 5H-J, 12H) consistent with a role for Zng1 in the promotion of Metap1 activity in vivo.

Finally, to test if Metap1 can compensate for Metap2-inhibition in WT animals but not in zng1 deficient animals (Frottin et al., 2006; Xiao et al., 2010), the METAP2 specific inhibitor TNP-470 was applied by immersion and larval survival was assessed. Similar to what was observed following treatment with Bengamide B, zng1” larvae displayed decreased survival compared to WT animals following TNP-470 exposure which became more pronounced in Zn-deplete conditions (FIGS. 5K, 121). Cumulatively, these data suggest that Metap1 activity is perturbed in zng1 mutant larvae and, consequently, the pharmacologic potency of both Metap inhibitors is enhanced in the absence of Zng1, particularly during Zn deprivation.

### Zng1-1-Mice are Sensitive to Dietary Zn Starvation and Display Signs of Mitochondrial Dysfunction.

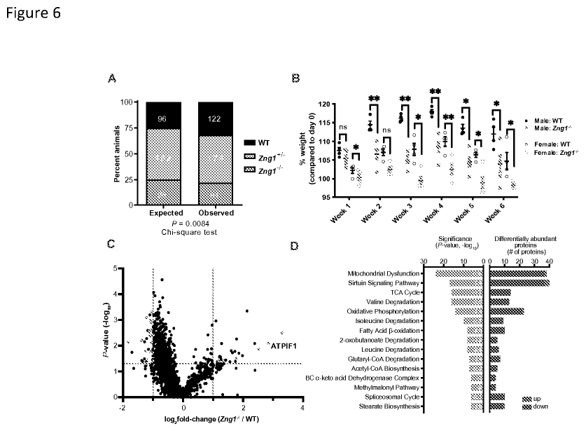

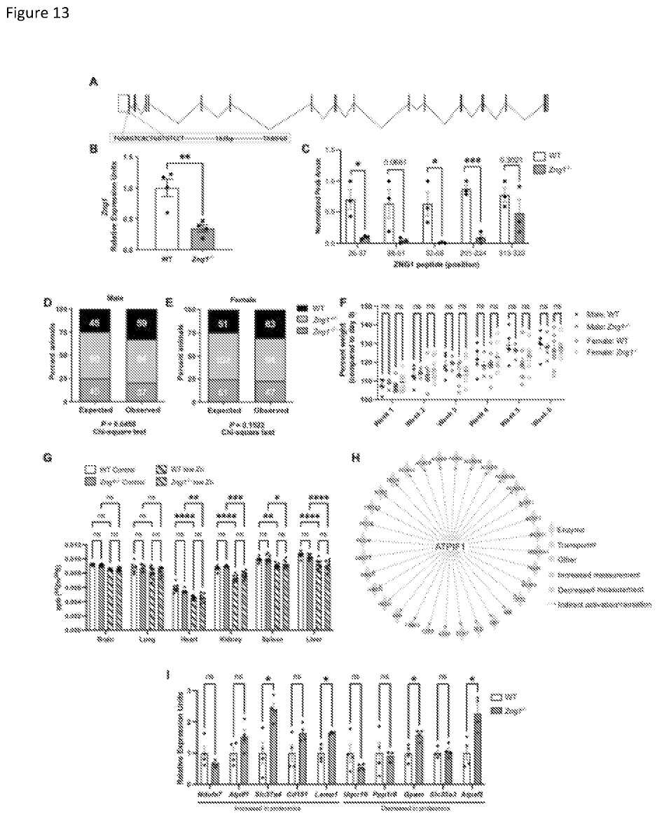

To investigate the impact of ZNG1 in a mammalian model, Zng1 mutant mice (hereafter referred to as Zng1^{-/-}) were generated using CRISPR/Cas9. The resulting deletion spanning the annotated start codon as well as 50 amino acids within exon 1 (including the CPELVPI motif) was subsequently confirmed at the transcriptional and protein level (FIG. 13A-C). Zng1^{-/-} animals were viable and did not display anomalies in gross morphology. Nevertheless, breeding patterns showed non-mendelian inheritance (FIGS. 6A and 13D, E), suggesting that ZNG1 functions in early development. We next assessed if ZNG1 effects Zn homeostasis during conditions of dietary Zn deprivation. WT and Zng1^{-/-} mice were administered a low Zn diet for 6 weeks and monitored for weight gain and organ specific Zn levels (FIG. 6B). Mice deficient in Zng1 displayed decreased weight gain when fed a low Zn diet (FIG. 6B) yet no difference in food uptake was observed. Notably, both WT and Zng1^{-/-}animals gained weight at similar rates when fed a control diet (FIG. 13F). While a low Zn diet was shown to effectively decrease Zn levels in various organ systems, differences in organ Zn levels between WT and mutant animals were not observed (FIG. 13G). These data suggest that loss of Zng1 leads to failure to access existing Zn pools, rather than affecting organ-specific total Zn levels.

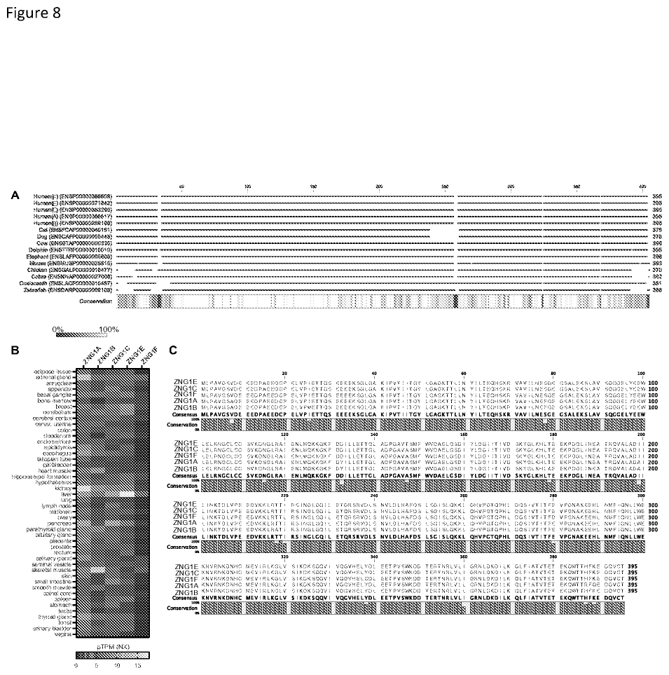

Due to the importance of METAP1 for maintaining cellular proteostasis (Hu et al., 2006; Jonckheere et al., 2018), the impact of Zng1 deficiency on the global proteome in Zn-deplete conditions was evaluated from WT and Zng1^{-/-} animals fed a low Zn diet for 5 weeks. Kidneys were selected for this analysis because ZNG1 proteins are highly expressed in human renal tissue (FIG. 8) (Uhlen et al., 2015). Sixty-three differentially abundant proteins were identified in the Zng1 mutant compared to the WT control (+2-fold cutoff, FIG. 6C, FIG. 17). Analysis of predicted localizations of these proteins using the UniProt database (UniProt, 2021) suggested that a large number localize to the mitochondria, particularly those with decreased abundance (FIG. 6C, D). Ingenuity Pathway Analysis (IPA) was performed to predict cellular processes affected by loss of Zng1 and revealed that numerous affected proteins play roles in canonical mitochondrial pathways (FIG. 6D) (Kramer et al., 2014). Notably, IPA also revealed that loss of Zng1 in Zn-starved animals significantly affects proteins connected to mitochondrial dysfunction, further suggesting negative effects on mitochondrial integrity. While most of the mitochondrial proteins were decreased in abundance in mutant animals (FIG. 6C, D), some proteins in this group were significantly elevated in Zng1^{-/-} mice. One of the proteins with the largest increase in abundance (+7.2-fold) in the mutant animals was the mitochondrial ATPase inhibitory factor 1 (IF1, ATPIF1) (Garcia-Bermudez and Cuezva, 2016). ATPIF1 is considered the master regulator of ATP synthase, and blocks both ATP synthesis and hydrolysis activities (Chen et al., 2014). Subsequent IPA upstream regulator analysis (URA) (Kramer et al., 2014) identified ATPIF as a main regulatory node (P-value of overlap=2.4E-33) with experimental linkage to a subset of (mitochondrial) proteins identified as being negatively affected by loss of ZNG1 (FIG. 13H). To evaluate if changes in protein abundance were a result of altered protein processing, transcriptomic analysis of a subset of the most differentially abundant proteins was performed. These data revealed modest changes in transcript levels, with some changes reaching statistical significance, indicating effects of Zng1 deficiency at both the transcript and protein level (FIG. 13I). Together, these results suggest that ZNG1 is important for maintaining mitochondrial function and organismal homeostasis during Zn starvation.

### Zng1 Promotes Cellular Proliferation and Respiration in Zn-Deplete Conditions.

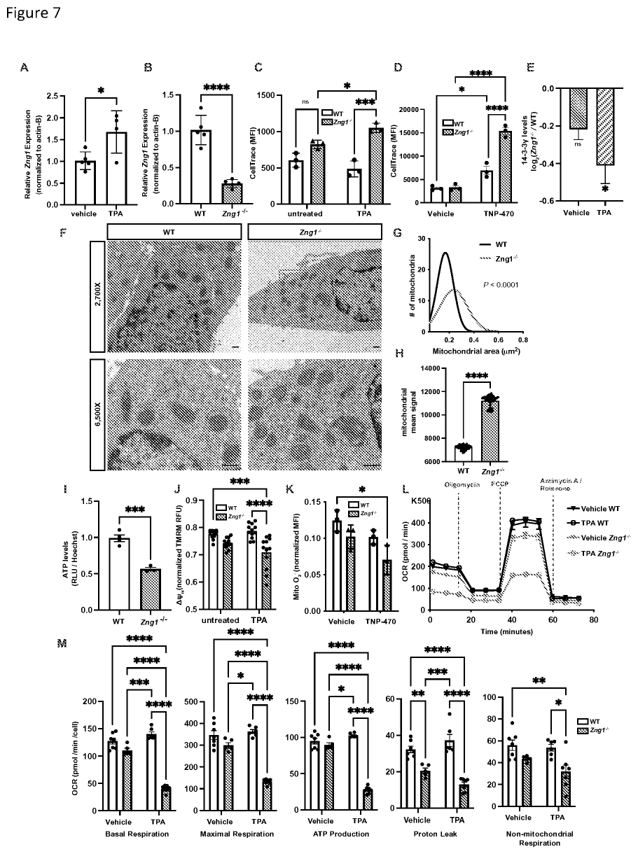

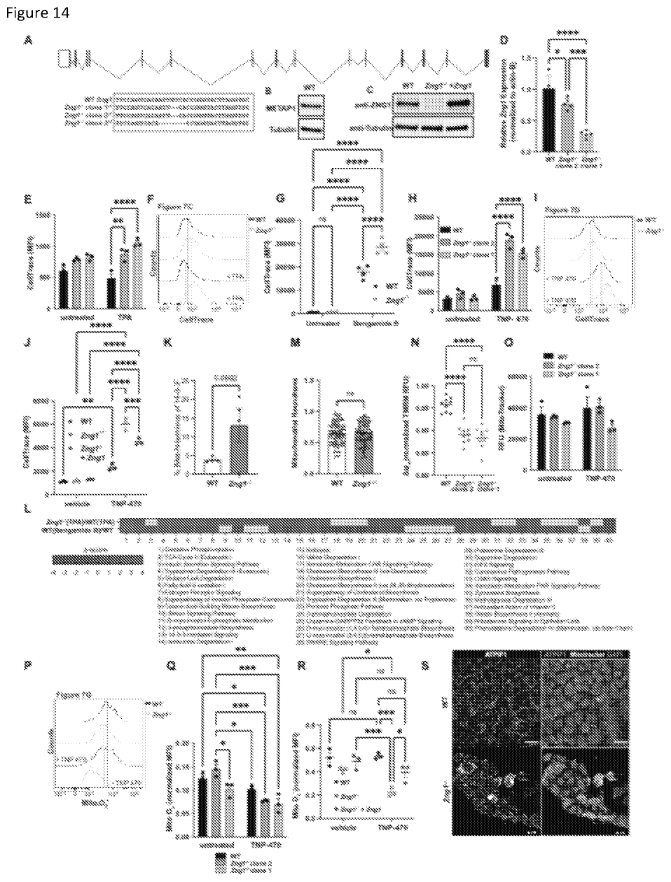

Given that Zng1 mutant mice displayed signs of impaired Zn homeostasis as well as disruption of mitochondrial function, a cell culture model was generated to further characterize the role of ZNG1. Murine proximal tubular epithelial cells (TKPTS) were selected as a model since mitochondrial dysfunction was detected in murine Zng1^{-/-} renal tissue, and TKPTS cell lines express both ZNG1 and METAP1 (FIGS. 7A, B and 14A-C). Zng1 mutant TKPTS cells (FIG. 14A) were generated using CRISPR/Cas9 and decreased mRNA expression (FIGS. 7B and 14D) and protein abundance (FIG. 14C) of ZNG1 were confirmed. The expression of Zng1 in TKPTS cells was dependent on Zn availability (FIG. 7A). While Zng1^{-/-} and WT cells grew at similar rates in untreated media, growth in the presence of Zn chelator tris(2-pyridylmethyl)amine (TPA) significantly slowed proliferation of the Zng1 mutant cells compared to WT as measured by increased retention of CellTrace dye (FIGS. 7C and 14E, F). Of note, exposure of WT TKPTS cells to the METAP1/2 inhibitor Bengamide B similarly reduced cellular proliferation, thereby phenocopying the growth impairment observed in Zng1 mutant cells in Zn-deplete conditions (FIG. 14G). Because ZNG1 supports proper function of METAP1 in vitro (FIG. 4), we postulated that Zng1-deficient cells would display increased sensitivity to METAP inhibition. Due to the overlapping substrate specificity of METAP1 and METAP2, TKPTS cells were exposed to the METAP2-specific inhibitor TNP-470. This strategy increases cellular reliance on METAP1 thereby permitting the evaluation of ZNG1-dependent METAP1 activity. Indeed, Zng1^{-/-} cells exhibited slowed proliferation relative to WT when exposed to TNP-470 (FIGS. 7D and 14H, I) and complementation with full-length Zng1 significantly enhanced proliferation of Zng1 mutant cells (FIG. 14J). Notably, decreased proliferation and increased sensitivity to METAP-inhibition have been reported in METAP1-deficient HAP1 cells (Jonckheere et al., 2018), which is consistent with the possibility that defects observed in Zng1^{-/-} cells may in part be mediated by dysregulated METAP1 activity.

To further assess a potential impairment of METAP1 function in the Zng1^{-/-} background, the abundance and iMet-status of the known METAP-substrate 14-3-3y were probed though proteomic profiling (Towbin et al., 2003). Analysis of WT and Zng1^{-/-} cells grown in Zn-replete and Zn-limiting (TPA-treated conditions) conditions revealed reduced levels of 14-3-3y in Zng1 mutant cells (FIG. 7E). We hypothesized that this reduction was a result of impaired METAP1-dependent iMet removal, and accordingly detected a trend towards higher iMet retention at the 14-3-3y N-terminus in TPA treated Zng1^{-/-} mutant cells as compared to WT (FIG. 14K). Taken together, these data are indicative of a reduction of METAP1 activity in Zng1^{-/-}cells. To further test if the proteomic changes in the Zng1 mutant resemble those of METAP-deficient cells, proteomic analysis was performed on WT cells treated with the METAP1/2 inhibitor Bengamide B and the results compared to the TPA-treated Zng1 mutant cells (FIG. 17). IPA was performed and significantly affected pathways in both datasets determined. Subsequent comparison of the two analyses showed remarkable overlap of the pathways affected by loss of Zng1 and pharmacological inhibition of METAP (FIG. 14L). Notably, pathways significantly enriched in both datasets included oxidative phosphorylation, TCA cycle (FIG. 14L), and mitochondrial dysfunction (P-value (−log 10) of 3 and 10.5 for Zng1^{-/-} and Bengamide B-treated, respectively). Collectively, these findings demonstrate that mutation of Zng1 phenocopies a METAP-deficient system, and that mitochondrial processes and function are affected by loss of ZNG1.

The reduced proliferation of Zng1 mutant cells (FIG. 7C,D), in combination with proteomic signatures of mitochondrial distress in Zng1^{-/-}cell lines and mice (FIGS. 6C and 14L) are consistent with a restriction in mitochondrial energy production. Since mitochondrial morphology reflects function (Picard et al., 2013), Zn starved WT and Zng1 mutant cells were imaged using transmission electron microscopy (TEM). Zng1 mutant cells were morphologically distinct (FIG. 7F) and displayed a significant change in mitochondrial cross-sectional area (FIG. 7G) in the absence of alterations of mitochondrial roundness, suggesting that mitochondria from Zng1^{-/-} cells are swollen (Figure S7M). Additionally, mitochondria of Zng1^{-/-}cells displayed increased electron density (FIG. 7H). Similar alterations in mitochondrial morphology have previously been associated with uncoupling of the mitochondrial membrane potential from ATP synthesis (Hackenbrock et al., 1971; Huet et al., 2018; Weissert et al., 2021). Consistent with this, cellular ATP levels quantified from the lysates of Zng1-TKPTS cells were decreased compared to WT (FIG. 7I). Together, these data suggest that ZNG1 contributes to maintaining mitochondrial metabolic homeostasis, and that a loss of ZNG1 renders cells less capable of sustaining the energy necessary for growth during conditions of stress.

To assess broad changes in oxidative phosphorylation, mitochondrial membrane potential was quantified using tetramethylrhodamine methyl ester (TMRM) at a quenching concentration where a decrease in fluorescence intensity correlates to an increase in membrane potential (Monteith et al., 2013; Scaduto and Grotyohann, 1999). TMRM fluorescence was decreased in Zng1^{-/-}cells compared to WT cells in conditions of Zn starvation, indicating mitochondria within Zng1 mutant cells are hyperpolarized (FIGS. 7J and 14N), consistent with reduced ATP synthase activity. Mitochondrial superoxide (O₂⁻) production, a byproduct of electron leakage during electron shuttling across the inner mitochondrial membrane (Forman and Kennedy, 1974; Loschen et al., 1974), was quantified using the fluorescent dye, MitoSOX. Mitochondrial O₂⁻ levels in Zng1 mutant cells were lower compared to WT cells, and this phenotype is exacerbated upon METAP2 inhibition (FIGS. 7K and S70-R). These results suggest that electron shuttling is decreased in Zng1 mutant cells despite hyperpolarization of the mitochondria.

To further interrogate the implications of loss of Zng1 on oxidative phosphorylation, the oxygen consumption rate (OCR) of WT and Zng1^{-/-} cells grown in media alone or in the presence of TPA was quantified by extracellular flux analysis. While Zng1^{-/-} cells exhibited a trend towards decreased basal and maximal respiration compared to WT cells, pre-treatment of mutant cells with TPA significantly impaired basal and maximal respiration, ATP production, and proton leak (FIG. 7L, M). Notably, Zng1^{-/-} cells showed accumulated ATPIF1 puncta colocalizing with mitochondrial structures (FIG. 14S), consistent with proteomic analysis of Zng1 mutant animals (FIG. 6C, D). These results support a model whereby loss of Zng1 negatively effects METAP1 activity, particularly during Zn starvation. In turn, impaired Metap1-dependent processing of the proteome likely disrupts mitochondrial homeostasis. Specifically, Zng1 mutant cells display hyperpolarization of the mitochondrial membrane with decreased oxidative phosphorylation, consistent with ATPIF1-dependent inhibition of ATP synthase. Taken together, these findings demonstrate that ZNG1 activates METAP1 activity and ZNG1 plays a critical role in regulating mitochondrial function and energy metabolism during Zn starvation.

### DISCUSSION

Zn is an essential structural and enzymatic cofactor for many proteins in all branches of life. Consequently, vertebrates have developed sophisticated systems for Zn uptake, intracellular distribution into organelles, and efflux to maintain organismal and cellular Zn homeostasis (Eide, 2006). While numerous aspects of Zn transport and trafficking have been well characterized, mechanisms for allocating Zn within the cell and to individual proteins remain largely unexplored. Distribution of this essential nutrient to “high priority” (Eide, 2006) proteins has been attributed to hypothetical Zn metallochaperones. However, functionally validated interactions with target proteins and bona fide Zn metallochaperone function have not been reported. In this study, evolutionarily divergent systems were used to establish a role for ZNG1 in regulating cellular Zn homeostasis, identifying METAP1 as a conserved client of ZNG1 and providing mechanistic insights into ZNG1-dependent METAP1 activation. Specifically, the ZNG1 N-terminus and METAP1 zf-C6H2 were identified as the primary interacting domains and structurally characterized. Upon interaction, METAP1 activates the GTPase activity specifically of Zn-bound ZNG1. GTP hydrolysis drives Zn transfer from ZNG1 into the METAP1 catalytic site either through the energy provided by phosphodiester bond cleavage or a conformational change upon hydrolysis, thereby metalating and activating METAP1. Importantly, the Zn binding affinities of ZNG1 and METAP1 are similar, suggesting that Zn transfer may have some element of kinetic control; alternatively, GDP modestly lowers the affinity of ZNG1 for Zn, facilitating capture by METAP1. In any case, the Zn- and GTP-dependent activation of METAP1 by ZNG1 is consistent with the assignment of ZNG1 as a metallochaperone.

The ZNG1-METAP1 interactions described in this study are likely to be more broadly conserved across eukaryotes. Consistent with our findings, this interaction was also suggested by a large-scale screen of the human interactome (Huttlin et al., 2015) and identified by a recent study that investigated Zng1p in Saccharomyces cerevisiae (Pasquini et al.). The ZNG1-METAP1 interaction in vertebrates is mediated by a conserved N-terminal CPELVPI motif in ZNG1 and the zf-C6H2 domain of METAP1. Our solution structure of the interaction domain reveals that the conserved features, including the hydrophobic binding pockets and the electrostatic surface of METAP1 required for ZNG1 binding, may well be shared among other ZNG1 clients or binding partners. Further sequence analysis reveals that approximately half of all eukaryotic ZNG1 homologs (Edmonds et al., 2021) share an N-terminal “[D/E]ₙψP×LVp” motif, in which a series of negatively charged residues [E/D], are followed by a non-aromatic hydrophobic residue (w), and a conserved proline, leucine, and valine (FIG. 3F). Our structure is fully consistent with this revised motif, and therefore likely captures key features that describe an evolutionarily conserved ZNG1-METAP1 interaction. Indeed, the N-terminus of S. cerevisiae Zng1p lacks the exact vertebrate CPELVPI motif, but matches the more general “[D/E]ₙψP×LVp” pattern. Although the sequence identity of the yeast and mouse Map1p/METAP1 N-terminal domains is less than 40% (Figure S2E), AlphaFold2 modeling of the Zng1p-Mapp interaction predicts a nearly identical fold and similar peptide interface (Jumper et al., 2021) (Figure S3I), where positive charges surround a hydrophobic groove and engage negatively charged ZNG1 peptides in both complexes. These findings suggest that our structure of the ZNG1-METAP1 interaction domain may be representative of how evolutionarily distant ZNG1 homologs interact with their targets.

The structure and revised ZNG1 motif also provide insight into what proteins may compete with ZNG1 to perform its metallochaperone function. While many proteins contain a more general P×L×P sequence, very few match the “[D/E]ₙψP×LVp” pattern identified here. One notable candidate is in the C-terminus of the β-subunit of the nascent polypeptide-associated complex (NAC), which protrudes from the ribosome exit tunnel (Gamerdinger et al., 2019; Lin et al., 2020). This motif likely explains how the zf-C6H2 domain localizes METAP1 to the ribosome in the presence of NAC (Nyathi and Pool, 2015; Vetro and Chang, 2002). The high affinity of ZNG1 for METAP1 may enable it to outcompete NAC under conditions of low cellular Zn.

Our zebrafish and mouse models of Zng1 deficiency link ZNG1 to mitochondrial function, organismal development, and Zn homeostasis. The connection between METAP1 and mitochondrial integrity is not without precedent. A previous study that utilized transcriptomic and proteomic approaches to characterize the effects of METAP1 loss in human HAP1 cells found changes to mitochondrial transcript and protein levels as a result of METAP1 mutation (Jonckheere et al., 2018). Such data offer a potential explanation as to how mutation of Zng1 in TKPTS cells results in mitochondrial dysfunction and attenuated cellular respiration, and ultimately explain the detrimental effects observed in Zng/deficient animals. Collectively, these findings highlight the importance of ZNG1 for survival during Zn limitation, likely by ensuring functional METAP1-dependent NME and maintaining animal energy balance during such conditions.

In conclusion, we established the vertebrate COG0523 ZNG1 protein family as a major feature of the vertebrate adaptive response to severe Zn deprivation. Using biochemical, structural, genetic, and pharmacological approaches across evolutionarily divergent models, including zebrafish and mice, we demonstrate a critical role for ZNG1 proteins in regulating cellular Zn homeostasis. Collectively, these data reveal the existence of a family of Zn metallochaperones and place ZNG1 at the center of a new paradigm for intracellular Zn trafficking. While these data show a clear physical interaction between ZNG1 and the metalloprotein METAP1, we also provide structural insights into how ZNG1 could interact with other targets. These findings reveal an evolutionary conserved strategy to ensure targeted Zn distribution to a network of high-priority client proteins during conditions of severe Zn starvation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Addlagatta, A., Hu, X., Liu, J. O., and Matthews, B.W. (2005). Structural basis for the functional differences between type I and type II human methionine aminopeptidases. Biochemistry 44, 14741-14749.
Andreini, C., Banci, L., Bertini, I., and Rosato, A. (2006). Counting the zinc-proteins encoded in the human genome. J Proteome Res 5, 196-201.
Arfin, S. M., and Bradshaw, R.A. (1988). Cotranslational processing and protein turnover in eukaryotic cells. Biochemistry 27, 7979-7984.
Ba, L. A., Doering, M., Burkholz, T., and Jacob, C. (2009). Metal trafficking: from maintaining the metal homeostasis to future drug design. Metallomics 1, 292-311.
Basu, A. K. (2018). DNA Damage, Mutagenesis and Cancer. Int J Mol Sci 19.
Brayer, K. J., Kulshreshtha, S., and Segal, D. J. (2008). The protein-binding potential of C2H2 zinc finger domains. Cell Biochem Biophys 51, 9-19.
Chen, W. W., Birsoy, K., Mihaylova, M. M., Snitkin, H., Stasinski, I., Yucel, B., Bayraktar, E. C., Carette, J. E., Clish, C. B., Brummelkamp, T. R., et al. (2014). Inhibition of ATPIF1 ameliorates severe mitochondrial respiratory chain dysfunction in mammalian cells. Cell Rep 7, 27-34.
Coneyworth, L. J., Jackson, K. A., Tyson, J., Bosomworth, H. J., van der Hagen, E., Hann, G. M., Ogo, O. A., Swann, D. C., Mathers, J. C., Valentine, R. A., et al. (2012). Identification of the human zinc transcriptional regulatory element (ZTRE): a palindromic protein-binding DNA sequence responsible for zinc-induced transcriptional repression. J Biol Chem 287, 36567-36581.
Cox, J., Hein, M. Y., Luber, C. A., Paron, I., Nagaraj, N., and Mann, M. (2014). Accurate proteome-wide label-free quantification by delayed normalization and maximal peptide ratio extraction, termed MaxLFQ. Mol Cell Proteomics 13, 2513-2526.
Crooks, G. E., Hon, G., Chandonia, J. M., and Brenner, S.E. (2004). WebLogo: a sequence logo generator. Genome Res 14, 1188-1190.
Delaglio, F., Grzesiek, S., Vuister, G. W., Zhu, G., Pfeifer, J., and Bax, A. (1995). NMRPipe: a multidimensional spectral processing system based on UNIX pipes. J Biomol NMR 6, 277-293.
Devirgiliis, C., Zalewski, P. D., Perozzi, G., and Murgia, C. (2007). Zinc fluxes and zinc transporter genes in chronic diseases. Mutat Res 622, 84-93.
Eddy, S. R. (2009). A new generation of homology search tools based on probabilistic inference. Genome Inform 23, 205-211.
Edmonds, K. A., Jordan, M. R., and Giedroc, D. P. (2021). COG0523 proteins: a functionally diverse family of transition metal-regulated G3E P-loop GTP hydrolases from bacteria to man. Metallomics 13.
Eide, D. J. (2006). Zinc transporters and the cellular trafficking of zinc. Biochim Biophys Acta 1763, 711-722.
Emilio Quinoa, M. A., Phillip Crews, and Gerald J. Bakus (1986). Bengamides, heterocyclic anthelmintics from a Jaspidae marine sponge. The Journal of Organic Chemistry 51 4494-4497.
Essner, J. (2016). Zebrafish embryo microinjection: Ribonucleoprotein delivery using the Alt-R CRISPR-Cas9 System. Coralville, Integrated DNA Technologies.
Fang, H., and Gough, J. (2013). DcGO: database of domain-centric ontologies on functions, phenotypes, diseases and more. Nucleic Acids Res 41, D536-544.
Fedotova, A. A., Bonchuk, A. N., Mogila, V. A., and Georgiev, P. G. (2017). C2H2 Zinc Finger Proteins: The Largest but Poorly Explored Family of Higher Eukaryotic Transcription Factors. Acta Naturae 9, 47-58.
Fischer Walker, C., and Black, R. E. (2004). Zinc and the risk for infectious disease. Annu Rev Nutr 24, 255-275.
Forman, H. J., and Kennedy, J. A. (1974). Role of superoxide radical in mitochondrial dehydrogenase reactions. Biochem Biophys Res Commun 60, 1044-1050.
Frottin, F., Martinez, A., Peynot, P., Mitra, S., Holz, R. C., Giglione, C., and Meinnel, T. (2006). The proteomics of N-terminal methionine cleavage. Mol Cell Proteomics 5, 2336-2349.
Gamerdinger, M., Kobayashi, K., Wallisch, A., Kreft, S. G., Sailer, C., Schlomer, R., Sachs, N., Jomaa, A., Stengel, F., Ban, N., et al. (2019). Early Scanning of Nascent Polypeptides inside the Ribosomal Tunnel by NAC. Mol Cell 75, 996-1006 e1008.
Garcia-Bermudez, J., and Cuezva, J. M. (2016). The ATPase Inhibitory Factor 1 (IF1): A master regulator of energy metabolism and of cell survival. Biochim Biophys Acta 1857, 1167-1182.
Gawron, D., Ndah, E., Gevaert, K., and Van Damme, P. (2016). Positional proteomics reveals differences in N-terminal proteoform stability. Mol Syst Biol 12, 858.
Gibbs, D.J., Bacardit, J., Bachmair, A., and Holdsworth, M. J. (2014). The eukaryotic N-end rule pathway: conserved mechanisms and diverse functions. Trends Cell Biol 24, 603-611.
Gibson, D. G. (2011). Enzymatic assembly of overlapping DNA fragments. Methods Enzymol 498, 349-361.
Gibson, D. G., Young, L., Chuang, R. Y., Venter, J.C., Hutchison, C. A., 3rd, and Smith, H. O. (2009). Enzymatic assembly of DNA molecules up to several hundred kilobases. Nat Methods 6, 343-345.
Giglione, C., Boularot, A., and Meinnel, T. (2004). Protein N-terminal methionine excision. Cell Mol Life Sci 61, 1455-1474.
Gross, C. T., and McGinnis, W. (1996). DEAF-1, a novel protein that binds an essential region in a Deformed response element. EMBO J 15, 1961-1970.
Guilbert, J. J. (2003). The world health report 2002-reducing risks, promoting healthy life. Educ Health (Abingdon) 16, 230.
Guntert, P. (2004). Automated NMR structure calculation with CYANA. Methods Mol Biol 278, 353-378.
Haas, C. E., Rodionov, D. A., Kropat, J., Malasarn, D., Merchant, S. S., and de Crecy-Lagard, V. (2009). A subset of the diverse COG0523 family of putative metal chaperones is linked to zinc homeostasis in all kingdoms of life. BMC Genomics 10, 470.
Hackenbrock, C. R., Rehn, T. G., Weinbach, E. C., and Lemasters, J. J. (1971). Oxidative phosphorylation and ultrastructural transformation in mitochondria in the intact ascites tumor cell. J Cell Biol 51, 123-137.
HaileMariam, M., Eguez, R. V., Singh, H., Bekele, S., Ameni, G., Pieper, R., and Yu, Y. (2018). S-Trap, an Ultrafast Sample-Preparation Approach for Shotgun Proteomics. J Proteome Res 17, 2917-2924.
Harter, M. R., Liu, C.D., Shen, C. L., Gonzalez-Hurtado, E., Zhang, Z. M., Xu, M., Martinez, E., Peng, C. W., and Song, J. (2016). BS69/ZMYND11 C-Terminal Domains Bind and Inhibit EBNA2. PLOS Pathog 12, e1005414.
Hilty, C., Wider, G., Fernandez, C., and Wuthrich, K. (2003). Stereospecific assignments of the isopropyl methyl groups of the membrane protein OmpX in DHPC micelles. J Biomol NMR 27, 377-382.
Howe, K. L., Achuthan, P., Allen, J., Allen, J., Alvarez-Jarreta, J., Amode, M. R., Armean, I. M., Azov, A. G., Bennett, R., Bhai, J., et al. (2021). Ensembl 2021. Nucleic Acids Res 49, D884-D891.
Hu, X., Addlagatta, A., Lu, J., Matthews, B. W., and Liu, J. O. (2006). Elucidation of the function of type 1 human methionine aminopeptidase during cell cycle progression. Proc Natl Acad Sci USA 103, 18148-18153.
Huang, Z., Zhang, X. A., Bosch, M., Smith, S. J., and Lippard, S. J. (2013). Tris(2-pyridylmethyl)amine (TPA) as a membrane-permeable chelator for interception of biological mobile zinc. Metallomics 5, 648-655.
Huet, D., Rajendran, E., van Dooren, G. G., and Lourido, S. (2018). Identification of cryptic subunits from an apicomplexan ATP synthase. Elife 7.
Huttlin, E. L., Ting, L., Bruckner, R. J., Gebreab, F., Gygi, M. P., Szpyt, J., Tam, S., Zarraga, G., Colby, G., Baltier, K., et al. (2015). The BioPlex Network: A Systematic Exploration of the Human Interactome. Cell 162, 425-440.
Hyberts, S. G., Milbradt, A. G., Wagner, A. B., Arthanari, H., and Wagner, G. (2012). Application of iterative soft thresholding for fast reconstruction of NMR data non-uniformly sampled with multidimensional Poisson Gap scheduling. J Biomol NMR 52, 315-327.
Hyberts, S. G., Takeuchi, K., and Wagner, G. (2010). Poisson-gap sampling and forward maximum entropy reconstruction for enhancing the resolution and sensitivity of protein NMR data. J Am Chem Soc 132, 2145-2147.
Ingerman, E., and Nunnari, J. (2005). A continuous, regenerative coupled GTPase assay for dynamin-related proteins. Methods Enzymol 404, 611-619.
Jao, L. E., Wente, S. R., and Chen, W. (2013). Efficient multiplex biallelic zebrafish genome editing using a CRISPR nuclease system. Proc Natl Acad Sci USA 110, 13904-13909.
Jonckheere, V., Fijalkowska, D., and Van Damme, P. (2018). Omics Assisted N-terminal Proteoform and Protein Expression Profiling On Methionine Aminopeptidase 1 (MetAP1) Deletion. Mol Cell Proteomics 17, 694-708.
Jordan, M.R., Wang, J., Weiss, A., Skaar, E. P., Capdevila, D. A., and Giedroc, D. P. (2019). Mechanistic Insights into the Metal-Dependent Activation of Zn(II)-Dependent Metallochaperones. Inorg Chem 58, 13661-13672.
Jumper, J., Evans, R., Pritzel, A., Green, T., Figurnov, M., Ronneberger, O., Tunyasuvunakool, K., Bates, R., Zidek, A., Potapenko, A., et al. (2021). Highly accurate protein structure prediction with AlphaFold. Nature 596, 583-589.
Kabir-ud-Din, B., M., Khan, I. A. (2003). Kinetics and mechanism of the ninhydrin reaction with DL-methionine in the absence and the presence of organic solvents. Indian J Chem 42B, 1132-1136.
Kateb, F., Perrin, H., Tripsianes, K., Zou, P., Spadaccini, R., Bottomley, M., Franzmann, T. M., Buchner, J., Ansieau, S., and Sattler, M. (2013). Structural and functional analysis of the DEAF-1 and BS69 MYND domains. PLOS One 8, e54715.
Kim, H. K., Kim, R. R., Oh, J. H., Cho, H., Varshavsky, A., and Hwang, C. S. (2014). The N-terminal methionine of cellular proteins as a degradation signal. Cell 156, 158-169.
Kramer, A., Green, J., Pollard, J., Jr., and Tugendreich, S. (2014). Causal analysis approaches in Ingenuity Pathway Analysis. Bioinformatics 30, 523-530.
Krezel, A., and Maret, W. (2017). The Functions of Metamorphic Metallothioneins in Zinc and Copper Metabolism. Int J Mol Sci 18.
Kumar, S., Stecher, G., Suleski, M., and Hedges, S. B. (2017). TimeTree: A Resource for Timelines, Timetrees, and Divergence Times. Mol Biol Evol 34, 1812-1819.
Kuzmic, P. (1996). Program DYNAFIT for the analysis of enzyme kinetic data: application to HIV proteinase. Anal Biochem 237, 260-273.
Lee, W., Tonelli, M., and Markley, J. L. (2015). NMRFAM-SPARKY: enhanced software for biomolecular NMR spectroscopy. Bioinformatics 31, 1325-1327.
Leopoldini, M., Russo, N., and Toscano, M. (2007). Which one among Zn(II), Co(II), Mn(II), and Fe(II) is the most efficient ion for the methionine aminopeptidase catalyzed reaction? J Am Chem Soc 129, 7776-7784.
Li, X., and Chang, Y. H. (1995). Amino-terminal protein processing in Saccharomyces cerevisiae is an essential function that requires two distinct methionine aminopeptidases. Proc Natl Acad Sci USA 92, 12357-12361.
Lin, Z., Gasic, I., Chandrasekaran, V., Peters, N., Shao, S., Mitchison, T. J., and Hegde, R. S. (2020). TTC5 mediates autoregulation of tubulin via mRNA degradation. Science 367, 100-104.
Liu, Y., Chen, W., Gaudet, J., Cheney, M. D., Roudaia, L., Cierpicki, T., Klet, R. C., Hartman, K., Laue, T. M., Speck, N. A., et al. (2007). Structural basis for recognition of SMRT/N-CoR by the MYND domain and its contribution to AML1/ETO's activity. Cancer Cell 11, 483-497.
Livak, K. J., and Schmittgen, T. D. (2001). Analysis of relative gene expression data using real-time quantitative PCR and the 2 (-Delta Delta C (T)) Method. Methods 25, 402-408.
Lo, M. N., Damon, L. J., Wei Tay, J., Jia, S., and Palmer, A. E. (2020). Single cell analysis reveals multiple requirements for zinc in the mammalian cell cycle. Elife 9.
Lohr, F., Hansel, R., Rogov, V. V., and Dotsch, V. (2007). Improved pulse sequences for sequence specific assignment of aromatic proton resonances in proteins. J Biomol NMR 37, 205-224.
Loschen, G., Azzi, A., Richter, C., and Flohe, L. (1974). Superoxide radicals as precursors of mitochondrial hydrogen peroxide. FEBS Lett 42, 68-72.
Maciejewski, M.W., Schuyler, A.D., Gryk, M.R., Moraru, II, Romero, P.R., Ulrich, E.L., Eghbalnia, H.R., Livny, M., Delaglio, F., and Hoch, J.C. (2017). NMRbox: A Resource for Biomolecular NMR Computation. Biophys J 112, 1529-1534.
MacLean, B., Tomazela, D.M., Shulman, N., Chambers, M., Finney, G.L., Frewen, B., Kern, R., Tabb, D.L., Liebler, D.C., and MacCoss, M.J. (2010). Skyline: an open source document editor for creating and analyzing targeted proteomics experiments. Bioinformatics 26, 966-968.
Meinnel, T., and Giglione, C. (2008). Tools for analyzing and predicting N-terminal protein modifications. Proteomics 8, 626-649.
Mirdita, M., Ovchinnikov, S., and Steinegger, M. (2021). ColabFold-Making protein folding accessible to all. bioRxiv, 2021.2008.2015.456425.
Monteith, A., Marszalec, W., Chan, P., Logan, J., Yu, W., Schwarz, N., Wokosin, D., and Hockberger, P. (2013). Imaging of mitochondrial and non-mitochondrial responses in cultured rat hippocampal neurons exposed to micromolar concentrations of TMRM. PLOS One 8, e58059.
Mortensen, B. L., Rathi, S., Chazin, W. J., and Skaar, E.P. (2014). Acinetobacter baumannii response to host-mediated zinc limitation requires the transcriptional regulator Zur. J Bacteriol 196, 2616-2626.
Muhire, B. M., Varsani, A., and Martin, D.P. (2014). SDT: a virus classification tool based on pairwise sequence alignment and identity calculation. PLOS One 9, e108277.
Nyathi, Y., and Pool, M. R. (2015). Analysis of the interplay of protein biogenesis factors at the ribosome exit site reveals new role for NAC. J Cell Biol 210, 287-301.
O'Halloran, T. V., and Culotta, V. C. (2000). Metallochaperones, an intracellular shuttle service for metal ions. J Biol Chem 275, 25057-25060.
Ogo, O. A., Tyson, J., Cockell, S. J., Howard, A., Valentine, R. A., and Ford, D. (2015). The zinc finger protein ZNF658 regulates the transcription of genes involved in zinc homeostasis and affects ribosome biogenesis through the zinc transcriptional regulatory element. Mol Cell Biol 35, 977-987.
Parichy, D. M., Elizondo, M. R., Mills, M. G., Gordon, T. N., and Engeszer, R. E. (2009). Normal table of postembryonic zebrafish development: staging by externally visible anatomy of the living fish. Dev Dyn 238, 2975-3015.
Pelton, J. G., Torchia, D. A., Meadow, N. D., and Roseman, S. (1993). Tautomeric states of the active-site histidines of phosphorylated and unphosphorylated IIIGlc, a signal-transducing protein from Escherichia coli, using two-dimensional heteronuclear NMR techniques. Protein Sci 2, 543-558.
Peroutka Iii, R.J., Orcutt, S. J., Strickler, J. E., and Butt, T. R. (2011). SUMO fusion technology for enhanced protein expression and purification in prokaryotes and eukaryotes. Methods Mol Biol 705, 15-30.
Picard, M., Shirihai, O. S., Gentil, B. J., and Burelle, Y. (2013). Mitochondrial morphology transitions and functions: implications for retrograde signaling? Am J Physiol Regul Integr Comp Physiol 304, R393-406.
Polevoda, B., and Sherman, F. (2003). N-terminal acetyltransferases and sequence requirements for N-terminal acetylation of eukaryotic proteins. J Mol Biol 325, 595-622.
Prasad, A.S. (2013). Discovery of human zinc deficiency: its impact on human health and disease. Adv Nutr 4, 176-190.
Rachineni, K., Arya, T., Singarapu, K.K., Addlagatta, A., and Bharatam, J. (2015). Chemical shift assignments of zinc finger domain of methionine aminopeptidase 1 (MetAP1) from Homo sapiens. Biomol NMR Assign 9, 351-353.
Raghunath, A., and Perumal, E. (2018). Analysis of Lethality and Malformations During Zebrafish (Danio rerio) Development. Methods Mol Biol 1797, 337-363.
Robert, X., and Gouet, P. (2014). Deciphering key features in protein structures with the new ENDscript server. Nucleic Acids Res 42, W320-324.
Rosenzweig, A. C. (2002). Metallochaperones: bind and deliver. Chem Biol 9, 673-677.
Sattler, M., Schleucher J., Griesinger C. (1999). Heteronuclear multidimensional NMR experiments for the structure determination of proteins in solution employing pulsed field gradients. Progress in Nuclear Magnetic Resonance Spectroscopy 34, 93-158.
Scaduto, R.C., Jr., and Grotyohann, L. W. (1999). Measurement of mitochondrial membrane potential using fluorescent rhodamine derivatives. Biophys J 76, 469-477.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Sheffield, P., Garrard, S., and Derewenda, Z. (1999). Overcoming expression and purification problems of RhoGDI using a family of “parallel” expression vectors. Protein Expr Purif 15, 34-39.
Shen, Y., and Bax, A. (2015). Protein structural information derived from NMR chemical shift with the neural network program TALOS-N. Methods Mol Biol 1260, 17-32.
Sigel, A., Sigel, H., and Sigel, R. K. O. (2013). Interrelations between essential metal ions and human diseases (Dordrecht; New York: Springer).
Stetefeld, J., McKenna, S. A., and Patel, T.R. (2016). Dynamic light scattering: a practical guide and applications in biomedical sciences. Biophys Rev 8, 409-427.
Sydor, A. M., Jost, M., Ryan, K. S., Turo, K. E., Douglas, C. D., Drennan, C. L., and Zamble, D. B. (2013). Metal binding properties of Escherichia coli YjiA, a member of the metal homeostasis-associated COG0523 family of GTPases. Biochemistry 52, 1788-1801.
Towbin, H., Bair, K. W., DeCaprio, J. A., Eck, M. J., Kim, S., Kinder, F. R., Morollo, A., Mueller, D. R., Schindler, P., Song, H. K., et al. (2003). Proteomics-based target identification: bengamides as a new class of methionine aminopeptidase inhibitors. J Biol Chem 278, 52964-52971.
Tyanova, S., Temu, T., Sinitcyn, P., Carlson, A., Hein, M.Y., Geiger, T., Mann, M., and Cox, J. (2016). The Perseus computational platform for comprehensive analysis of (prote) omics data. Nat Methods 13, 731-740.
Uhlen, M., Fagerberg, L., Hallstrom, B.M., Lindskog, C., Oksvold, P., Mardinoglu, A., Sivertsson, A., Kampf, C., Sjostedt, E., Asplund, A., et al. (2015). Proteomics. Tissue-based map of the human proteome. Science 347, 1260419.
Ulrich, E.L., Akutsu, H., Doreleijers, J.F., Harano, Y., Ioannidis, Y.E., Lin, J., Livny, M., Mading, S., Maziuk, D., Miller, Z., et al. (2008). BioMagResBank. Nucleic Acids Res 36, D402-408.
UniProt, C. (2021). UniProt: the universal protein knowledgebase in 2021. Nucleic Acids Res 49, D480-D489.
Varshavsky, A. (2011). The N-end rule pathway and regulation by proteolysis. Protein Sci 20, 1298-1345.
Vetro, J. A., and Chang, Y.H. (2002). Yeast methionine aminopeptidase type 1 is ribosome-associated and requires its N-terminal zinc finger domain for normal function in vivo. J Cell Biochem 85, 678-688.
Vetro JA, D.B., and Chang Y (2004). Methionine Aminopeptidase. Emerging role in angiogenesis. In Aminopeptidases in Biology and Disease, H.a. Lendeckel, ed. (New York: Kluwer Academic/Plenum Publishers).
Weissert, V., Rieger, B., Morris, S., Arroum, T., Psathaki, O.E., Zobel, T., Perkins, G., and Busch, K.B. (2021). Inhibition of the mitochondrial ATPase function by IF1 changes the spatiotemporal organization of ATP synthase. Biochim Biophys Acta Bioenerg 1862, 148322.
Wellenreuther, G., Cianci, M., Tucoulou, R., Meyer-Klaucke, W., and Haase, H. (2009). The ligand environment of zinc stored in vesicles. Biochem Biophys Res Commun 380, 198-203.
White, K.N., Tenney, K., and Crews, P. (2017). The Bengamides: A Mini-Review of Natural Sources, Analogues, Biological Properties, Biosynthetic Origins, and Future Prospects. J Nat Prod 80, 740-755.
Wong, A., Vallender, E.J., Heretis, K., Ilkin, Y., Lahn, B.T., Martin, C. L., and Ledbetter, D.H. (2004). Diverse fates of paralogs following segmental duplication of telomeric genes. Genomics 84, 239-247.
Xiao, Q., Zhang, F., Nacev, B.A., Liu, J. O., and Pei, D. (2010). Protein N-terminal processing: substrate specificity of Escherichia coli and human methionine aminopeptidases. Biochemistry 49, 5588-5599.
Yamazaki T, F.-K.J., Kay LE (1993). Two-dimensional NMR experiments for correlating carbon-13.beta. and proton.delta./.epsilon. chemical shifts of aromatic residues in ¹³C-labeled proteins via scalar couplings. J Am Chem Soc 115, 11054-11055.
Zougman, A., Selby, P. J., and Banks, R.E. (2014). Suspension trapping (STrap) sample preparation method for bottom-up proteomics analysis. Proteomics 14, 1006-1000.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

1. A method of treating a disease or condition characterized by increased activity of a methionine aminopeptidase (METAP) protein, comprising interfering with a transfer of Zn to the METAP protein from a Zn-regulated GTPase-GPTase metalloprotein activator 1 (ZNG1) protein.

2. The method of claim 1, wherein the disease or condition is characterized by uncontrolled cellular proliferation.

3. The method of claim 2, wherein the disease or condition is a cancer.

4. The method of claim 1, wherein the disease or condition is a metabolic disease or condition.

5. The method of claim 4, wherein the disease or condition is obesity.

6. The method of claim 1, wherein the interfering comprises reducing or preventing the transfer of Zn from the ZNG1 protein to the METAP protein.

7. The method of claim 1, wherein the interfering comprises reducing or inhibiting an activity of the ZNG1 protein.

8. The method of claim 6, wherein the reducing or preventing the transfer of Zn comprises reducing or preventing an interaction between the ZNG1 protein and the METAP protein.

9. The method of claim 8, wherein the reducing or preventing an interaction is effected by a peptide comprising the amino acid sequence:
MAAVETRVCETDGCSSEAKLQCPTCIKLGIQGSYFCSQECFKGSWATHKLLHKKAKDE K (SEQ ID NO:70).

10. The method of claim 1, wherein the METAP protein is METAP1.

11. The method of claim 1, further including administering one or more METAP2 inhibitors.

12. A method of reducing an activity of a methionine aminopeptidase (METAP) protein, comprising interfering with a transfer of Zn to the METAP protein from a Zn-regulated GTPase metalloprotein activator 1 (ZNG1) protein.

13. The method of claim 12, wherein the METAP protein is METAP1.

14. The method of claim 1, wherein the interfering comprises reducing or preventing the transfer of Zn from the ZNG1 protein to the METAP protein.

15. The method of claim 1, wherein the interfering comprises reducing or inhibiting an activity of the ZNG1 protein.

16. The method of claim 1, wherein the reducing or preventing the transfer of Zn comprises reducing or preventing an interaction between the ZNG1 protein and the METAP protein.

17. The method of claim 16, wherein the reducing or preventing an interaction is effected by a peptide comprising the amino acid sequence:
MAAVETRVCETDGCSSEAKLQCPTCIKLGIQGSYFCSQECFKGSWATHKLLHKKAKDE K (SEQ ID NO:70).
